(19) United States
(12) Patent Application Publication
(10) Pub. No.: US 2018/0355382 A1
(43) Pub. Date: Dec. 13, 2018

(54) LARGE GENOMIC DNA KNOCK-IN AND USES THEREOF

(71) Applicant: The Jackson Laboratory, Bar Harbor, ME (US)

(72) Inventors: David Bergstrom, Farmington, CT (US); Tiffany Leidy-Davis, Farmington, CT (US)

(21) Appl. No.: 15/968,943

(22) Filed: May 2, 2018

Related U.S. Application Data
(63) Continuation of application No. PCT/US2016/060788, filed on Nov. 7, 2016.
(60) Provisional application No. 62/252,080, filed on Nov. 6, 2015.

(51) Int. Cl. C12N 15/90; C12N 9/22; C12N 15/11; A01K 67/027

(52) U.S. Cl. CPC C12N 15/907; C12N 9/22; C12N 15/11; A01K 67/0278; A01K 2227/105; C12N 2310/20; A01K 2207/15; A01K 2217/072; C12N 2800/80

(57) ABSTRACT

The present invention provides compositions and methods for utilizing a large capacity cloning vector (e.g., BAC) to carry a large exogenous genomic DNA (about 10-300 kb) flanked by a proximal and distal regions (10 kb) to efficiently insert into the genome of a cell in a CRISPR/Cas9-stimulated homologous recombination. Methods and compositions for microinjecting a large human gene into a mouse zygote to prepare a genetically modified mouse are also provided.

### REFERENCE TO RELATED APPLICATION

This application is a continuation application of International Patent Application No. PCT/US2016/060788, filed on Nov. 7, 2016 and published as WO2017/079724, which claims the benefit of the filing date under 35 U.S.C. § 119(e) to U.S. Provisional Application No. 62/252,080, filed on Nov. 6, 2015, the entire contents of each of which are hereby incorporated by reference in their entirety.

### GOVERNMENT SUPPORT

The present invention was made with U.S. government support under Grant No. P30CA034196, awarded by the United States National Cancer Institute (NCI), of the National Institutes of Health (NIH). The U.S. government has certain rights in the invention.

### BACKGROUND OF THE INVENTION

Genome editing is a type of genetic engineering in which DNA is inserted, replaced, or removed from a genome. In recent years, genome editing has been achieved using artificially engineered nucleases (a/k/a “molecular scissors”). The nucleases create double-strand breaks (DSBs) at desired locations in the genome, and harness the cell's endogenous mechanisms to repair the induced break by natural processes of homology directed repair (HDR, a common form of which is homologous recombination (HR)) and nonhomologous end-joining (NHEJ).

Restriction endonucleases (REs) are often used to create DSBs in a target DNA. Because of the short recognition sequences (usually 4-8 bp), REs create too many DSBs in large genomic DNA regions. To overcome this problem, several distinct classes of nucleases have been bioengineered to generate site-specific DSBs more suitable for large genomic regions; for example, Zinc finger nucleases (ZFNs), Transcription Activator-Like Effector Nucleases (TALENs), the CRISPR/Cas system, and engineered meganuclease.

Meganucleases are commonly found in some microbial species. They possess the unique property of having very long recognition sequences (>14 bp), thus making them naturally specific, and suitable for generating site-specific DSBs during genome editing in large genomes. However, the limitation of this approach is that there are few known meganucleases, thus severely restricting the target sequences that can be covered by this method. Mutagenesis and high throughput screening methods have been used to create meganuclease variants that recognize unique sequences. Various meganucleases have been fused to create hybrid enzymes with new recognition sequences. Others have attempted to rationally design meganuclease by altering the DNA-interacting residues of the meganuclease in order to design sequence-specific meganucleases (See, e.g., U.S. Pat. No. 8,021,867).

Other traditional genetic engineering technologies in genome editing include random transgenesis, targeted transgenesis, and recombinase-mediated cassette exchange (RMCE). Each of these methods has its drawbacks. For example, random transgenic methods deviate from genome modification at the cognate endogenous locus, sufficing to allow transgenes to integrate randomly (where they are subject to variegated expression). During targeted transgenesis, transgenes may be directed specifically to standardized safe harbor sites to limit this position-effect variegation but even here the transgenes are unlinked to their endogenous cognate genes. Like the related RMCE method, targeted transgenesis may involve the use of antibiotic selection cassettes flanked by recombinase-binding sites. In addition to the added complexity, deleting these selection cassettes requires breeding to specific recombinase-expressing mice, thereby prolonging strain development.

For traditional gene-targeting, of the sort in use in the mouse for the past thirty years, the traditional paradigm based on a large body of literature, has been to create plasmid vectors with two homology arms of a few to several kilobase pairs in length to act as donor molecules. These arms are situated within the plasmid so as to flank investigator-altered sequences that will be incorporated into the genome after introduction of the plasmid vector into embryonic stem (ES) cells and HDR. Positive and negative selection cassettes are frequently employed to aid in selecting the rare embryonic stem (ES) cell clones containing properly integrated sequences. This technique is sufficient for modifying genomic sequence on a scale from one nucleotide up to several thousand base pairs. The method may fall short, however, when attempting to alter entire mouse genes that often extend over 10s or 100s of thousands of base pairs.

As genome engineering tools, the CRISPR-Cas endonucleases serve as instruments for generating DNA double-strand breaks (DSBs) with locus-of-interest specificity, at high frequency, and across a wide variety of strains and organisms. When faced with DSBs, cells of the organism being perturbed respond with the NHEJ pathway and the HDR pathway to repair the DSBs. DSBs repaired by the more rapid and error-prone NHEJ pathway are characterized by the deletion or insertion of a small number of nucleotides (INDELS), which, when they are within the open reading frame of a protein of interest, may lead to hypomorphic or null mutations of the original gene of interest. In contrast, DSBs repaired by HDR, in the presence of a homologous template (e.g., sister chromatid, donor molecule), provide the opportunity to introduce precise DNA modifications into the organism, at the site of the DSB.

The CRISPR technique is rapidly expanding, and is applied across multiple species and different genome-editing fields. Despite advancing CRISPR technology, at present, however, to the best of Applicant's knowledge, HDR (e.g., HR) based recombination is limited to editing relatively small regions of genomic DNA. For example, in the Homologous Recombination (HR) FAQs section of Addgene website aimed to address technical issues when using HR for gene editing following DSB creation by CRISPR/Cas, a question was raised relating to how long each homology arm should be when attempting to use the CRISPR/Cas9 system to create specific mutations or insertions by Homologous Recombination (HR). The recommended approach, for introducing small mutations (e.g., those <50 bp) or a single-point mutation, is to use a single stranded DNA (ssDNA) oligo (as opposed to a plasmid) as the HR template for transfection into the target cell. The ssDNA oligo typically has around 100-150 bp of total homology, with the small or point mutation situated in the middle, thus giving about 50-75 bp of homology arm on each side of the mutation. For large changes (such as >100 bp insertions or deletions), a plasmid donor is typically used, with two homology arms of around 800 bp on each side flanking the desired insertion or mutation. The typical size of such a plasmid donor is approximately 5 kb (Yang et al., Cell 154(6):1370-1379, 2013). In response to questions concerning the maximum amount of DNA that can be inserted into the genome using CRISPR/Cas for Homologous Recombination (HR), and the length of the homology arms for efficient recombination, the Addgene website indicated that the longest length that has been attempted for insertion is 1 kb, with homology arms that were 800 bp in length.

The uncertainty and unpredictability about certain key aspects of the technology, such as the nature and suitability of the DSBs created by CRISPR/Cas, and the effect of homology arm length on CRISPR-associated HDR, especially when the insert size is larger than a few kilobases, may contribute to the fact that CRISPR-associated HDR has been so far limited to the insertion of small DNA fragments (e.g., from a single nucleotide to one or a few kilobases at most) into the host genome.

Accordingly, there is a continuing need to develop novel means to introduce large genomic DNA into a host genome via homologous recombination.

### SUMMARY OF THE INVENTION

In one aspect, the present invention provides a method of inserting a large exogenous genomic DNA via homologous recombination to replace an endogenous genomic DNA in the genome of a cell of a mammal, comprising the steps of:
(a) providing a bacterial artificial chromosome (BAC);
(b) providing a large exogenous genomic DNA of about 10-300 kb;
(c) inserting said large exogenous genomic DNA into said BAC,
   wherein said large exogenous genomic DNA is flanked by a proximal region of about 10-30 kb, and a distal region of about 10-30 kb, and
   wherein said proximal region and said distal region flank said endogenous genomic DNA in the genome of said cell;
(d) preparing a first pair of CRISPR/Cas9 guide RNAs (gRNAs), said first pair comprises a first gRNA and a second gRNA, wherein said first gRNA and said second gRNA target a first Cas9 cleavage site and a second Cas9 cleavage site, respectively, in the endogenous genomic DNA, within about 250 bp from a proximal junction where said proximal region joins said endogenous genomic DNA in the genome of the cell;
(e) preparing a second pair of CRISPR/Cas9 guide RNAs (gRNAs), said second pair comprises a third gRNA and a fourth gRNA, wherein said third gRNA and said fourth g RNA target a third Cas9 cleavage site and a fourth Cas9 cleavage site, respectively, in the endogenous genomic DNA, within about 250 bp from the distal junction where said distal region joins said endogenous genomic DNA in the genome of the cell;
(f) providing a Cas9 protein, or a Cas9 coding sequence capable of producing the Cas9 protein; and
(g) introducing into said cell of said mammal:
   (i) said BAC in step (c);
   (ii) said first pair of CRISPR/Cas9 guide RNAs in step (d);
   (iii) said second pair of CRISPR/Cas9 guide RNAs in step (e); and
   (iv) said Cas9 protein or Cas9 coding sequence in step (f);
      whereby,
      (i) said first pair of gRNAs directs said Cas9 protein to cleave said first and said second Cas9 cleavage sites in said endogenous genomic DNA at the proximal junction to generate a first double-stranded break (DSB);
      (ii) said second pair of gRNAs directs said Cas9 protein to cleave said third and said fourth Cas9 cleavage sites in said endogenous genomic DNA at the distal junction to generate a second DSB; and
      (iii) said large exogenous genomic DNA is integrated into the genome of the cell at said first DSB and said second DSB via homologous recombination to replace said endogenous genomic DNA between the proximal region and the distal region.

In certain embodiments, the large exogenous genomic DNA is about 15-200 kb; preferably about 20-100 kb; and more preferably about 25 kb.

In certain embodiments, the cell is a zygote. In certain embodiments, with respect to the zygote, step (g) is performed by microinjection. In certain embodiments, microinjection is performed using about 1-10 ng/μL of the BAC containing the large exogenous genomic DNA; preferably using about 2-8 ng/μL; more preferably using about 5 ng/μL.

In certain embodiments, the cell is an embryonic stem (ES) cell. In certain embodiments, with respect to ES cells, step (g) is performed by electroporation.

In certain embodiments, the BAC carries no selection marker.

In certain embodiments, the BAC is pBACe3.6, pBACGK1.1, pBACGMR, pBAC-red, pTARBAC1, pTARBAC1.3, pTARBAC2, pTARBAC2.1, pTARBAC3, pTARBAC4, or pTARBAC6.

In certain embodiments, the large exogenous genomic DNA is from a different strain of the same species of the mammal. In certain embodiments, the large exogenous genomic DNA is from a different species of the mammal.

In certain embodiments, the mammal is a mouse.

In certain embodiments, the first and the second Cas9 cleavage sites are independently within about 100 bp, 50 bp, or 10 bp from the proximal junction.

In certain embodiments, the first gRNA and the second gRNA bind different strands (i.e., plus and minus strands) of the endogenous genomic DNA.

In certain embodiments, the first and the second Cas9 cleavage sites are the two potential Cas9 cleavage sites closest to the proximal junction.

In certain embodiments, the third and the fourth Cas9 cleavage sites are independently within about 100 bp, 50 bp, or 10 bp from the distal junction.

In certain embodiments, the third gRNA and the fourth gRNA bind different strands (i.e., plus and minus strands) of the endogenous genomic DNA.

In certain embodiments, the third and the fourth Cas9 cleavage sites are the two potential Cas9 cleavage sites closest to the distal junction.

In certain embodiments, in step (f), the Cas9 protein is provided in a complex comprising the first gRNA, the second gRNA, the third gRNA, or the fourth gRNA.

In a related aspect, however, the present method can be carried out using only one of the first and the second gRNAs to create the first DSB.

In another related aspect, the present method can be carried out using only one of the third and the fourth gRNAs to create the second DSB.

In one aspect, the present invention provides a method of generating a non-human mammal whose cells harboring a large exogenous genomic that have replaced an endogenous genomic DNA via homologous recombination, and capable of transmitting the large exogenous genomic DNA through germline, comprising the steps of:
(a) providing a bacterial artificial chromosome (BAC);
(b) providing a large exogenous genomic DNA of about 10-300 kb;
(c) inserting said large exogenous genomic DNA into the BAC,
   wherein the large exogenous genomic DNA is flanked by a proximal region of about 10-30 kb, and a distal region of about 10-30 kb, and wherein the proximal region and the distal region flank the endogenous genomic DNA in the genome of the mammal;
(d) preparing a first pair of CRISPR/Cas9 guide RNAs (gRNAs), the first pair comprises a first gRNA and a second gRNA, wherein the first gRNA and the second gRNA target a first Cas9 cleavage site and a second Cas9 cleavage site, respectively, in the endogenous genomic DNA, within about 250 bp from a proximal junction where the proximal region joins the endogenous genomic DNA in the genome of the mammal;
(e) preparing a second pair of CRISPR/Cas9 guide RNAs (gRNAs), the second pair comprises a third gRNA and a fourth gRNA, wherein the third gRNA and the fourth g RNA target a third Cas9 cleavage site and a fourth Cas9 cleavage site, respectively, in the endogenous genomic DNA, within about 250 bp from the distal junction where the distal region joins the endogenous genomic DNA in the genome of the mammal;
(f) providing a Cas9 protein, or a Cas9 coding sequence capable of producing the Cas9 protein; and
(g) introducing into a zygote of the mammal:
   (i) the BAC in step (c);
   (ii) the first pair of CRISPR/Cas9 guide RNAs in step (d);
   (iii) the second pair of CRISPR/Cas9 guide RNAs in step (e); and
   (iv) the Cas9 protein or Cas9 coding sequence in step (f);
(h) preparing a pseudopregnant female of the same species of the mammal;
(j) implanting the zygote in step (g) into the pseudopregnant female to give birth to an offspring of the mammal.

In certain embodiments, the large exogenous genomic DNA is about 15-200 kb; preferably about 20-100 kb; and more preferably about 25 kb.

In certain embodiments, step (g) is performed with microinjection.

In certain embodiments, the mammal is a hemizygote or a homozygote with respect to the large exogenous genomic DNA. In certain embodiments, about 50% or 100% of the progeny of the mammal carry the large exogenous genomic DNA.

In certain embodiments, the present method further comprises, if necessary, generating a progeny of the mammal that is a hemizygote or a homozygote with respect to the large exogenous genomic DNA.

In certain embodiments, the mammal is a species where ES cell technology is lacking.

In certain embodiments, microinjection is performed using about 1-10 ng/μL of the BAC containing the large exogenous genomic DNA; preferably using about 2-8 ng/μL; more preferably using about 5 ng/μL.

In certain embodiments, the first and the second Cas9 cleavage sites are independently within about 100 bp, 50 bp, or 10 bp from the proximal junction.

In certain embodiments, the third and the fourth Cas9 cleavage sites are independently within about 100 bp, 50 bp, or 10 bp from the distal junction.

In certain embodiments, the first gRNA and the second gRNA bind different strands (i.e., plus and minus strands) of the endogenous genomic DNA.

In certain embodiments, the third gRNA and the fourth gRNA bind different strands (i.e., plus and minus strands) of the endogenous genomic DNA.

In certain embodiments, the first and the second Cas9 cleavage sites are the two potential Cas9 cleavage sites closest to the proximal junction.

In certain embodiments, the third and the fourth Cas9 cleavage sites are the two potential Cas9 cleavage sites closest to the distal junction.

In certain embodiments, in step (f), the Cas9 protein is provided in a complex comprising the first gRNA, the second gRNA, the third gRNA, or the fourth gRNA.

In a related aspect, however, the present method can be carried out using only one of the first and the second gRNAs to create the first DSB.

In another related aspect, the present method can be carried out using only one of the third and the fourth gRNAs to create the second DSB.

In another aspect, the present invention provides an artificial genomic DNA comprising: a central region of a large genomic DNA from a first organism, a proximal region of a genomic DNA from a second organism, and a distal region of a genomic DNA from the second organism, wherein the central region is flanked by the proximal region and the distal region.

Exemplary sizes of the central region are: 10 kb, 15 kb, 20 kb, 25 kb, 30 kb, 35 kb, 40 kb, 45 kb, 50 kb, 60 kb, 80 kb, 100 kb, 150 kb, 200 kb, 250 kb, 300 kb, or 350 kb.

The central region of the genomic DNA from the first organism replaces a homologous or corresponding central region of the second organism flanked by the proximal region and the distal region in the second organism.

Exemplary sizes of the homologous or corresponding central region of the second organism are: 10 kb, 15 kb, 20 kb, 25 kb, 30 kb, 35 kb, 40 kb, 45 kb, 50 kb, 60 kb, 80 kb, 100 kb, 150 kb, 200 kb, 250 kb, 300 kb, or 350 kb.

In certain embodiments, the length of the proximal region and the length of the distal region both are sufficiently long to support homologous recombination.

Exemplary sizes of the proximal region are: 10 kb, 15 kb, 20 kb, 25 kb, 30 kb, 35 kb, 40 kb, 45 kb, or at least about 50 kb. Exemplary sizes of the distal region are: 10 kb, 15 kb, 20 kb, 25 kb, 30 kb, 35 kb, 40 kb, 45 kb, or at least about 50 kb.

In certain embodiments, the homologous or corresponding central region is about 20 kb, the central region is about 25-30 kb, and the proximal region and the distal region are each about 10 kb.

In certain embodiments, the first organism and the second organism are the same species. In certain embodiments, the first organism and the second organism are different species. In certain preferred embodiments, the first organism is human, and the second organism is mouse (or rat).

In one aspect, the present invention provides a vector capable of carrying a large exogenous DNA and compatible for homologous recombination in accordance with the present invention.

In certain embodiments, the vector suitable for (CRISPR-created) double-stranded break (DSB)—homologous recombination (HR)-mediated knock-in in a zygote comprises any one of the subject artificial genomic DNA. In certain embodiments, the DSB is created by CRISPR/Cas or CRISPR/cpf1. In certain embodiments, the vector has no selectable marker.

In certain embodiments, the vector is suitable for homologous recombination in embryonic stem (ES) cells, the vector comprises any one of the subject artificial genomic DNA.

In certain embodiments, the vector is a plasmid, a Phage λ, a cosmid, a Bacteriophage P1 vector, a P1 artificial chromosome (PAC), a Bacterial artificial chromosome (BAC), or a Yeast Artificial Chromosomes (YAC). Preferably, the vector is a BAC. Exemplary BAC includes, but not limited to pBACe3.6, pBACGK1.1, pBACGMR, pBAC-red, pTARBAC1, pTARBAC1.3, pTARBAC2, pTARBAC2.1, pTARBAC3, pTARBAC4, pTARBAC6, or a modified version thereof.

In certain embodiments, the vector or coding sequence encoding the CRISPR/Cas9 is a CRISPR/Cas9 mRNA.

In a related aspect, the present invention provides a method of introducing the central region of the first organism in-between the proximal and the distal regions of the second organism as described herein, the method comprising introducing a subject vector into an ES cell under conditions that permit homologous recombination.

In certain embodiments, the present method further comprises transferring the ES cell or the zygote into a pseudo-pregnant female.

In certain embodiments, the present method further comprising genotyping the mammal arising from the microinjected zygote or progeny thereof. The genotyping can be used to verify the Cas9 binding sites of intact (or small INDEL-containing) host mammal alleles, the endogenous/exogenous genomic DNA junctions, and/or the breakpoints of any deletion-bearing alleles.

In certain embodiments, the present method further comprises sequencing amplification products from genotyping reactions.

In certain embodiments, the present method further comprising genetic mapping of the integrated large exogenous genomic DNA to verify integration at desired locus.

It should be understood that any one embodiment described herein, including those only disclosed in the examples or one section of the specification, is intended to be able to combine with any one or more other embodiments unless explicitly disclaimed.

### BRIEF DESCRIPTION OF THE DRAWINGS

FIG. 1 shows a general scheme for the construction of the Bcl2l11/BCL2L11 targeting vector/donor molecule. A gene-targeting vector/donor molecule was constructed placing a 25-kbp segment of the human BCL2L11 gene between mouse homology arms, placing removable selectable marker cassettes at each end of the human segment, and placing loxP sites around a 2.9-kbp segment of human DNA deleted in 12% of the East Asian population.

FIG. 2 shows the organization of genotyping primers for mouse (M), humanized (M/H), and deletion-bearing (ΔM) alleles of BCL2L11/Bcl2l11. Schematic showing the organization of genotyping primers. Numbers, primer designation as in Table 4; left and right segments of horizontal black lines, flanking regions of the mouse Bcl2l11 region; central segment of top horizontal black line, central (to be replaced) region of the mouse Bcl2l11 gene; blue line, central segment of human BCL2L11 gene.

FIGS. 3A and 3B show the result of linkage analysis of the BCL2L11 integration site following CRISPR-stimulated homologous recombination in mouse zygotes. Shown are the linkage analyses for 22 F2 progeny of a C57BL/6NJ×FVBB6NF1/J-BCL2L11 backcross (upper panel) and 28 F2 progeny of an FVB/NJ×FVBB6NF1/J-BCL2L11 backcross (lower panel). Linkage and haplotype analysis indicate that the BCL2L11 vector's integration has occurred between markers rs4223406 and rs3689600 and its segregation is fully concordant with markers rs13476756 and rs3662211. This result is entirely consistent with integration of the human BCL2L11 segment within the endogenous mouse Bcl2l11 gene as designed.

### DETAILED DESCRIPTION OF THE INVENTION

DEFINITIONS—the terms used in this application shall have the following meanings.

As used herein, the term “large exogenous genomic DNA” refers to a foreign genomic DNA with respect to the genome of a mammal, with the foreign genomic DNA having a length of at least about 10 kb, e.g., about 10-300 kb, about 15-200 kb, about 20-100 kb, or about 25-50 kb. For example, a 50 kb human genomic DNA is a large exogenous genomic DNA with respect to a mouse genome.

As used herein, the term “homologous recombination” refers to a type of genetic recombination in which nucleotide sequences are exchanged between two similar or identical molecules of DNA known as homologous sequences or homology arms. Homologous recombination often involves the following basic steps: after a double-strand break (DSB) occurs on both strands of DNA, sections of DNA around the 5′ ends of the DSB are cut away in a process called resection. In the strand invasion step that follows, an overhanging 3′ end of the broken DNA molecule “invades” a similar or identical (or homologous) DNA molecule, e.g., a homology arm, that is not broken. After strand invasion, the further sequence of events may follow either of two main pathways—the DSBR (double-strand break repair) pathway or the SDSA (synthesis-dependent strand annealing) pathway.

As used herein, the term “endogenous genomic DNA” refers to a certain segment of genomic DNA in a mammal that is to be replaced by the large exogenous genomic DNA as defined herein. The endogenous genomic DNA to be replaced or deleted may or may not be homologous in sequence to the large exogenous genomic DNA, so long as they are both flanked by the same homology arms. The endogenous genomic DNA is at least about 10 kb in length. It is the sequence homology (e.g., identity) between the proximal region joined to the endogenous genomic DNA and the proximal region joined to the large exogenous genomic DNA; and the sequence homology (e.g., identity) between the distal region joined to the endogenous genomic DNA and the distal region joined to the large exogenous genomic DNA, that allow homologous recombination to occur, preferably in the presence of DSBs at/near the proximal and distal junctions.

As used herein, a “proximal region” refers to a segment of a genomic DNA at least about 10 kb in length, that (1) joins one end of the endogenous genomic DNA in the genome of the mammal, (2) joins one end of the large exogenous genomic DNA on a homologous recombination targeting vector, and (3) serves as one of the two flanking homology arms that facilitate homologous recombination to replace the endogenous genomic DNA with the large exogenous genomic DNA in the genome of the mammal.

As used herein, the term “proximal junction” refers to the location where the proximal region joins the endogenous genomic DNA in the genome of the mammal.

As used herein, a “distal region” refers to a segment of a genomic DNA at least about 10 kb in length, that (1) joins the other end of the endogenous genomic DNA in the genome of the mammal, (2) joins the other end of the large exogenous genomic DNA on a homologous recombination targeting vector, and (3) serves as the other of the two flanking homology arms that facilitate homologous recombination to replace the endogenous genomic DNA with the large exogenous genomic DNA in the genome of the mammal.

As used herein, the term “distal junction” refers to the location where the distal region joins the endogenous genomic DNA in the genome of the mammal.

As used herein, the term “artificial genomic DNA” refers to an artificial genomic DNA created by joining one end of a large exogenous genomic DNA from a first mammal to a proximal region from a second mammal, and by joining the other end of the large exogenous genomic DNA to a distal region from the second mammal. The large exogenous genomic DNA, the proximal region, and the distal region are each at least about 10 kb in length.

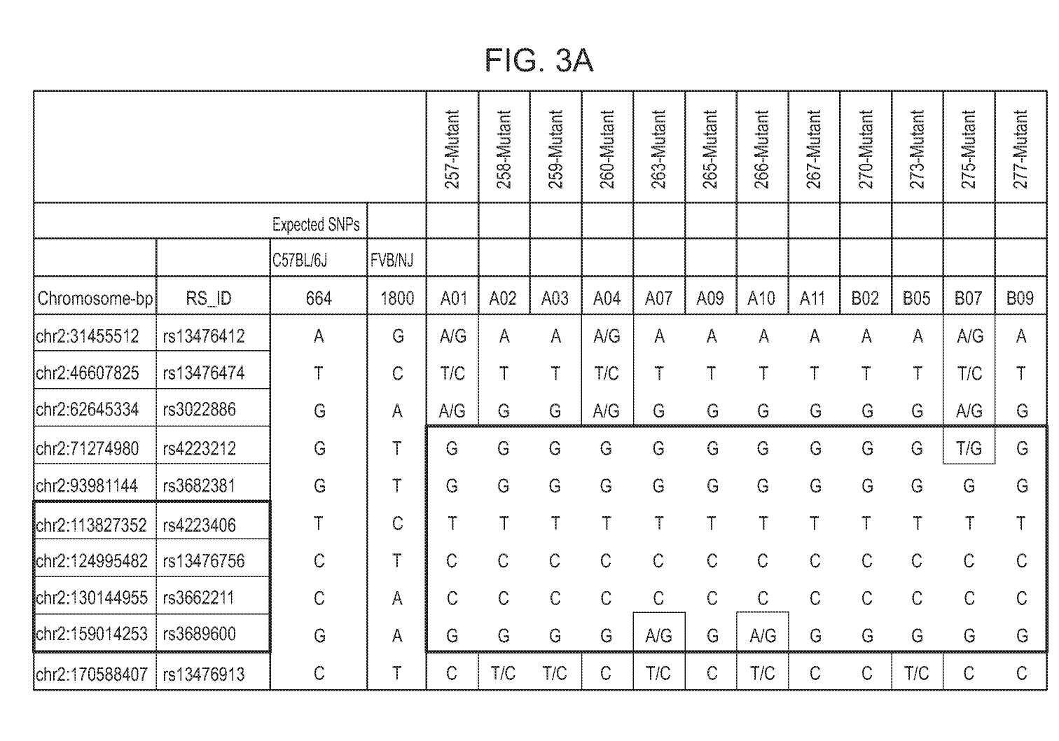

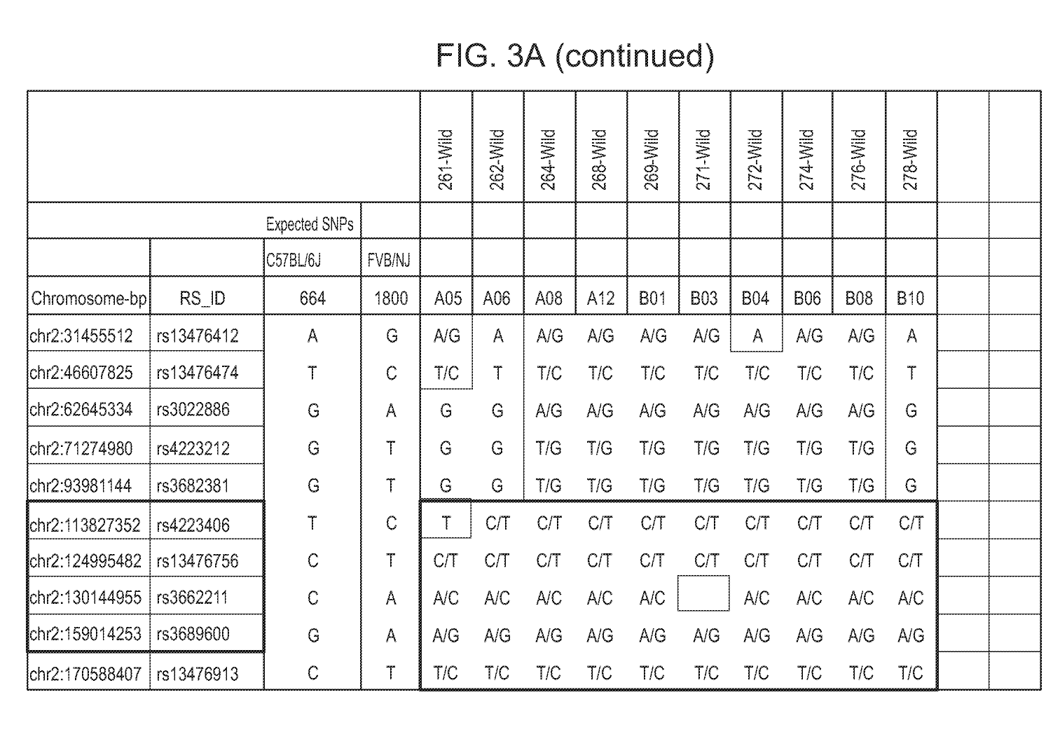

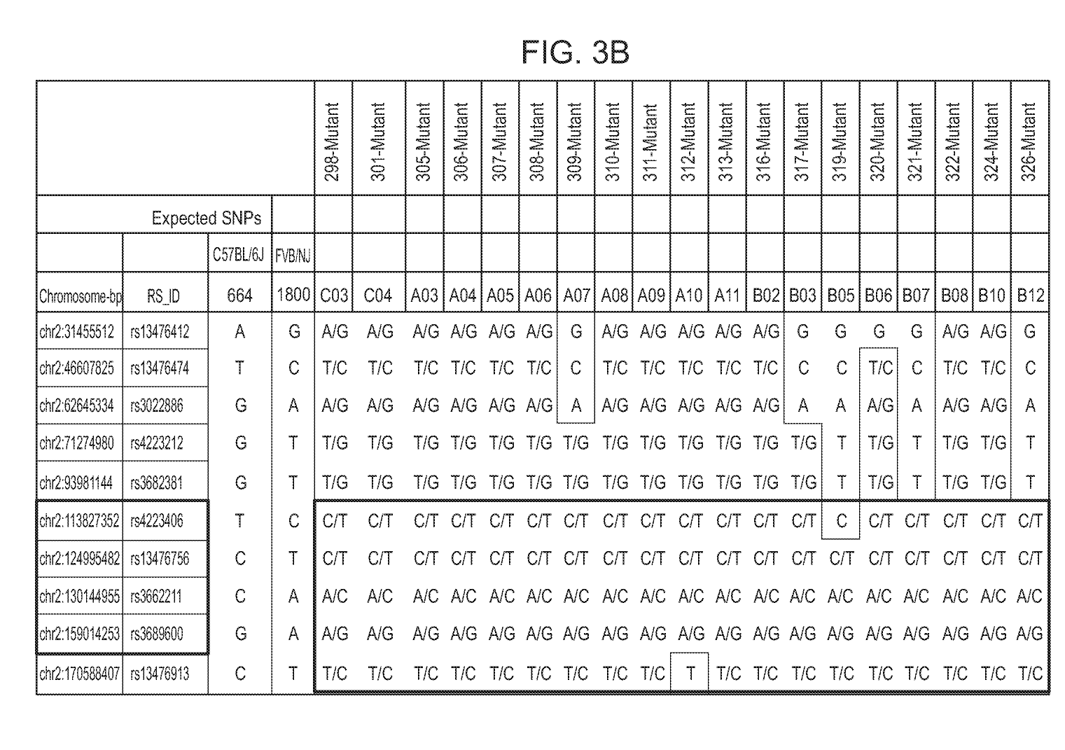

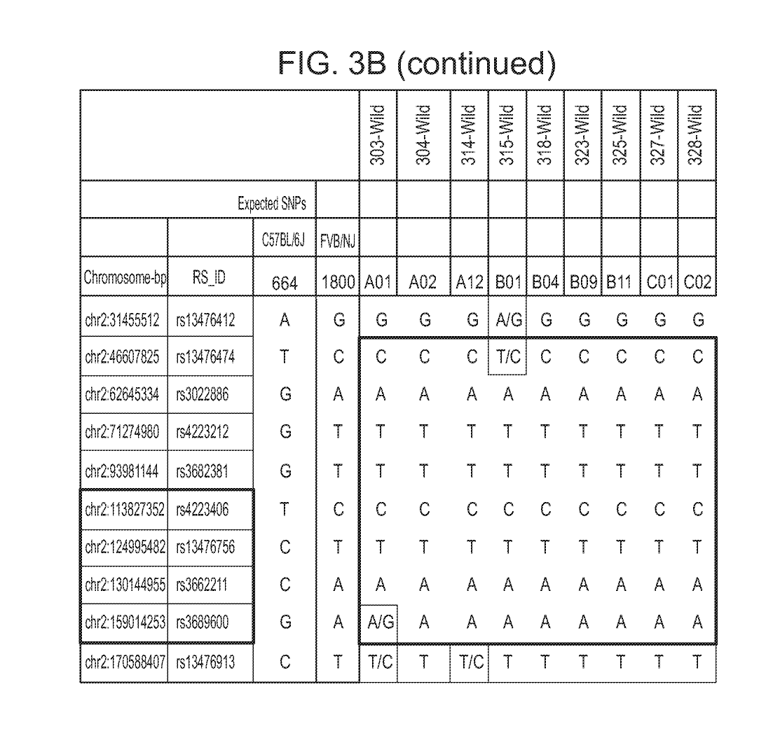

As used herein, the term “CRISPR associated protein 9” or “Cas9” protein refers to an RNA-guided DNA endonuclease associated with the CRISPR (Clustered Regularly Interspaced Short Palindromic Repeats) type II adaptive immunity system found in certain bacteria, such as Streptococcus pyogenes and other bacteria. For purposes of this application, Cas9 protein is not limited to the wild-type (wt) Cas9 found in Streptococcus pyogenes. It is intended to cover amino acids 7-166 or 731-1003 of the Cas9/Csnl amino acid sequence (of Streptococcus pyogenes), as depicted in FIG. 3 and SEQ ID NO: 8 of WO 2013/176772 (incorporated by reference); the corresponding portions in any one of the amino acid sequences SEQ ID NOs: 1-256 and 795-1346 of WO 2013/176772 (incorporated by reference); and the corresponding portions in any one of the amino acid sequences of the orthogonal Cas9 sequences from S. pyogenes, N. meningitidis, S. thermophilus and T. denticola (See, Esvelt et al., Nature Methods, 10(11): 1116-1121, 2013, incorporated by reference).

As used herein, the term “Cas9 coding sequence” refers to a polynucleotide capable of being transcribed and/or translated, according to a genetic code functional in a host cell/host mammal, to produce a Cas9 protein. The Cas9 coding sequence may be a DNA (such as a plasmid) or an RNA (such as an mRNA).

As used herein, the term “Cas9 riboprotein” refers to a protein/RNA complex consisting of Cas9 protein and an associated guide RNA.

As used herein, the term “embryonic stem (ES) cell” refers to a pluripotent stem cell derived from the inner cell mass (ICM) of a blastocyst (an early-stage preimplantation embryo of a mammal), that can be cultured after an extended periods in vitro, before it is inserted/injected into the cavity of a normal blastocyst, and be induced to resume a normal program of embryonic development to differentiate into all cell types of an adult mammal, including germ cells.

As used herein the term “ES cell technologies” refers to technologies developed for isolating, culturing, and manipulating ES cells, e.g., for gene transfer experiments. ES cell technologies are complex but powerful approaches to germline gene insertion, but they have thus far been established only in limited mammalian species, including mice, and to a lesser extent rat and human. Thus in most mammals, for which the instant CRISPR/Cas9-driven homologous recombination can be used to insert large exogenous genomic DNA, ES cell technologies are lacking.

As used herein, the term “zygote” refers to a eukaryotic cell formed by a fertilization event between two gametes, e.g., an egg and a sperm from a mammal.

As used herein, the term “zygosity” refers to the degree of similarity of the alleles for a trait in an organism.

As used herein, the term “homozygote” is used with respect to a particular gene or DNA (e.g., the large exogenous genomic DNA insertion into the host genome), and refers to a diploid cell or organism in which both homologous chromosomes have the same alleles or copies of the gene/DNA.

As used herein, the term “heterozygote” is used with respect to a particular gene or DNA (e.g., the large exogenous genomic DNA insertion into the host genome), and refers to a diploid cell or organism in which the two homologous chromosomes have different alleles/copies/versions of the gene or DNA.

As used herein, the term “hemizygote” is used with respect to a particular gene or DNA (e.g., the large exogenous genomic DNA insertion into the host genome), and refers to a diploid cell or organism in which an allele/copy/version of the gene or DNA is present in only one of the two homologous chromosomes (i.e., the gene or DNA is absent in the other homologous chromosome). Hemizygosity is observed when one copy of a gene is deleted, or in the heterogametic sex when a gene is located on a sex chromosome (e.g., on the X chromosome of a mammal). Hemizygosity is observed when an exogenous transgene is introduced into a locus on one chromosome, but is absent on the same locus on the other, homologous chromosome. However, a transgene can be bred to homozygosity and maintained as an inbred line if desirable and proper.

As used herein, the term “bacterial artificial chromosome (BAC)” refers to a large capacity DNA construct (typically 7 kb in length but is capable of containing an insert with a size of about 150-350 kbp) constructed based on a functional fertility plasmid (or F-plasmid of E. coli) and genomes of large DNA viruses (including those of baculovirus and murine cytomegalovirus), and used for transforming and cloning in bacteria, usually E. coli. A typical BAC has the following common components: repE (for plasmid replication and regulation of copy number); parA and parB (for partitioning F plasmid DNA to daughter cells during division and ensures stable maintenance of the BAC); T7 & Sp6 phage promoters for transcription of inserted genes; and an optional selectable marker for antibiotic resistance (some BACs also have lacZ at the cloning site for blue/white selection).

Accordingly, the present invention overcomes the disadvantages of the prior art by providing a BAC-based vector carrying a large exogenous genomic DNA for homologous recombination. The present invention provides a method of constructing a BAC-based vector. The BAC-based vectors of the present invention, when used in combination with CRISPR-Cas9, facilitate the efficient delivery of large genomic DNA to the genome of a target cell via homologous recombination.

The present invention described herein the partly based on the discovery that exogenous genomic DNAs of large size (e.g., about 10, 15, 20, 25, 30, 35, 50, 75, 100, 150, 200, 250, 300, or 350 kb) can be knocked-in to the genome of an organism. The present method utilizes homology arms (e.g., 10 kb-30 kb) suitable for homologous recombination flanking a large deletion/gap (e.g., about 10, 15, 20, 30, 35, 50, 75, 100, 150, 200, 250, 300, or 350 kb, etc.) in a target genome. The present method utilizes CRISPR/Cas9 components in combination with the homologous recombination.

The present invention as described and exemplified herein has numerous advantages, for example: 1) expanding the physical size of CRISPR-driven knock-ins and gene replacements to ≥25-kbp; 2) opening multiple strains and species to long range DNA modification; 3) obviating the need for antibiotic selection of embryonic stem (ES) cells; and 4) avoiding the recombinase-mediated excision of selection cassettes.

In one aspect, the present invention provides a large exogenous genomic DNA. The large exogenous genomic DNA is contained within a large capacity cloning vector for introduction into a host mammal in order to replace an endogenous genomic DNA, may be of large DNA sizes of about 10-300 kb, preferably between about 15-200 kb, and most preferably between about 100-150 kb.

The large exogenous genomic DNA can be human or non-human. For example, the non-human genomic DNA can be from an animal, a mammal (such as a non-human mammal), a rodent (e.g., a mouse, a rat, a hamster, a guinea pig, a rabbit, etc.), a yeast, a bacterium, and the like.

In certain embodiments, the large exogenous genomic DNA may contain a large foreign gene that encodes a protein, for example, a therapeutic protein (such as one that compensates for an inherited or acquired deficiency). Exemplary therapeutic proteins include: human growth hormone (rHGH), human insulin (BHI); follicle-stimulating hormone (FSH); Factor VIII; Factor IX; erythropoietin (EPO); granulocyte colony-stimulating factor (G-CSF); alpha-glactosidase A; alpha-L-iduronidase (rhIDU; laronidase); N-acetylgalactosamine-4-sulfatase (rhASB; galsulfase); Dornase alfa; tissue plasminogen activator (TPA); glucocerebrosidase; interferon (IF) Interferon-0; insulin-like growth factor 1 (IGF-1); and somatotropin, and the like.

Due to the large size of the large exogenous genomic DNA, it is possible to encode a series of different proteins with large promoter elements. For example, one can encode many proteins that together form a protein complex under cell-specific or exogenously regulated gene expression.

In certain embodiments, the large exogenous genomic DNA may encode a gene of interest (GOI), such as a gene a mutation of which has been linked to a disease or condition. The mutant gene may encode a mutant protein associated with a disease (e.g., cancer, neurodegenerative disease, autoimmune disease, inflammatory disease, etc). The integration of the foreign gene into a host genome provides unique functional genomic animal models (or assays), which can be useful to determine the presence or function of a gene in a particular genomic insert. For example, the gene may be human BCL2L11 (BCL2-like 11 apoptosis facilitator), and the mutation is a deletion of a portion of its intron 2. A mouse model in which the homologous mouse Bcl2l11 has been replaced by the human mutant BCL2L11 gene provides a valuable model to study the human disease associated with the human mutation.

In certain embodiments, the large exogenous genomic DNA may contain regulatory or controlling DNA sequences, including promoter, enhancer regions, or other transcriptional regulatory elements.

In certain embodiments, the large exogenous genomic DNA may comprise human or mammalian centromeric DNA for the creation of human or mammalian artificial chromosomes.

In another aspect, the present invention utilizes a large capacity cloning vector, such as a BAC, YAC and the like, to introduce the large exogenous genomic DNA into a host genome.

In certain embodiments, the present invention is directed to a BAC-based vector carrying a large exogenous genomic DNA, comprising: (a) a large capacity cloning vector, and (b) a large exogenous genomic DNA, wherein the BAC-based vector can deliver the large exogenous genomic DNA into a target cell.

Representative BAC includes pBACe3.6, pBACGK1.1, pBACGMR, pBAC-red, pTARBAC1, pTARBAC1.3, pTARBAC2, pTARBAC2.1, pTARBAC3, pTARBAC4, pTARBAC6, and the like.

BAC libraries and especially those containing human genomic DNA as a result of the Human Genome Project are readily available to those skilled in the art (See, e.g., Simon, Nature Biotechnol. 15:839, 1997).

In certain embodiments, the BAC vector has no selection marker. Such BAC vectors are suitable for methods of the present invention in which such vectors are microinjected into animal zygotes.

In certain embodiments, the BAC vector carries one or more selection marker genes. Such BAC vectors can be used for ES cells in which selection may be required. Suitable selection markers include, without limitation, neomycin resistant gene (e.g., Neo^{R}); Blasticidin S resistant gene (e.g., Bsd^{R}); puromycin resistant gene (puro^{R}), etc.

BAC is a preferred large capacity cloning vector, given the sizes of the inserts and the flanking homologous regions. However, other large capacity cloning vectors known to those skilled in the art, can also be used in the present invention. These include, e.g., cosmids (Evans et al., Gene 79:9-20, 1989), yeast artificial chromosomes (YACs) (Sambrook et al., A Molecular Cloning: A Laboratory Manual, 2^{nd} Edition, Cold Spring Harbor Press, Cold Spring Harbor, N.Y., 1989), mammalian artificial chromosomes (MACs) (Vos et al., Nature Biotechnology 15:1257-1259, 1997), human artificial chromosomes (Harrington et al., Nature Genetics 15: 345-354, 1997), or viral-based vectors, such as, CMV, EBV, or baculovirus based vectors.

In another aspect, the present invention provides an improved and simplified method for converting large exogenous genomic DNA in a large capacity DNA cloning vectors, such as, a bacterial artificial chromosome (BAC) clone, to a relatively smaller capacity cloning vector (such as a plasmid). This allows the large exogenous genomic DNA within the large capacity DNA cloning vector (e.g., BAC) more efficiently or more easily delivered to a target cell, and expressed in vitro or in vivo.

Thus in certain embodiments, the vector is a plasmid capable of carrying inserts up to about 15 kb. The plasmid may contain an origin of replication for replicating inside a prokaryote (e.g., a bacterium) independently of the host chromosome. The plasmid may also be able to replicate in a eukaryotic cell. The plasmid can also carry a selective marker, such as a gene for antibiotic resistance, that allows for the selection of cells containing the plasmid. The plasmid may further carry a reporter gene or marker gene to label or identify cell clones containing the plasmid.

In certain embodiments, the vector is a modified Phage λ, which is a double-stranded DNA virus. The wildtype λ chromosome is 48.5 kb long, and can be modified by replacing non-essential viral sequences in the λ chromosome with inserts of up to about 25 kb, leaving only phage genes required for formation of viral particles and infection. The insert DNA can be replicated with the viral DNA and be packaged together into viral particles for efficient infection of and multiplication within a host cell.

In certain embodiments, the vector is a cosmid vector that contains a small region of bacteriophage λ DNA known as the cos sequence, which allows the cosmid to be packaged into bacteriophage λ particles. Cosmids are capable of carrying inserts of up to 45 kb in size. Particles containing a linearized cosmid can be introduced into a host cell by transduction.

In certain embodiments, the vector is a Bacteriophage P1 vector that can carry inserts of between 70-100 kb in size. Such vectors begin as linear DNA molecules packaged into bacteriophage P1 particles. These particles are then injected into a bacterial host, such as an E. coli strain, that expresses Cre recombinase. The linear P1 vector becomes circularized by recombination between two loxP sites in the vector. The P1 vector may contains a gene for antibiotic resistance. The P1 vector may contain a (positive) selection marker to distinguish clones containing an insert from those that do not. The P1 vector may contain a P1 plasmid replicon to ensure that only one copy of the vector is present in a cell. The P1 vector can alternatively contain a P1 lytic replicon that is controlled by an inducible promoter, which allows the amplification of more than one copy of the vector per cell.

In certain embodiments, the vector is a P1 artificial chromosome (PAC) having features of both P1 vectors and Bacterial Artificial Chromosomes (BACs). Similar to P1 vectors, PACs contain a plasmid and a lytic replicon as described above. Unlike P1 vectors, they do not need to be packaged into bacteriophage particles for transduction. Instead they are introduced into E. coli as circular DNA molecules through electroporation as BACs are. The PACs can carry inserts of between 130-150 kb in size.

In certain embodiments, the vector is a Yeast Artificial Chromosomes (YAC), which are linear DNA molecules containing the necessary features of an authentic yeast chromosome, including telomeres, a centromere, and an origin of replication. Large inserts of DNA can be ligated into the middle of the YAC so that there is an arm of the YAC on either side of the insert. The recombinant YAC can be introduced into yeast by transformation. The YAC may comprise a selectable marker to allow for the identification of YAC-containing transformants Theoretically there is no upper limit on the size of insert a YAC can hold. In practice, YACs usually hold inserts of about 250-2000 kb, typically inserts of about 250-400 kb in size.

The present invention is further directed to a method of constructing BAC-based vector, by subcloning a large genomic DNA into a BAC. Methods of subcloning are well known in the art. More specifically, methods of moving large DNA inserts from one large capacity cloning vector into another large capacity cloning vector have been described (Wade-Martins et al., Nucl. Acids Res. 27:1674-1682, 1999; Wade-Martins et al., Nature Biotechnol. 18:1311-1314, 2000).

In another aspect, the present invention provides an artificial genomic DNA construct comprising a large exogenous genomic DNA from a first organism as a central region, a proximal region of a genomic DNA from a second organism, and a distal region of the genomic DNA from the second organism, wherein the large exogenous DNA is flanked by the proximal region and the distal region.

In certain embodiments, the proximal region and the length of the distal region are both sufficiently long to support homologous recombination.

Exemplary sizes of the large exogenous genomic DNA/central region are: 10 kb, 15 kb, 20 kb, 25 kb, 30 kb, 35 kb, 40 kb, 45 kb, 50 kb, 60 kb, 80 kb, 100 kb, 150 kb, 200 kb, 250 kb, 300 kb, or 350 kb.

The large exogenous genomic DNA/central region of the genomic DNA from the first organism replaces a homologous or corresponding central region of the second organism flanked by the proximal and distal regions in the second organism.

In certain embodiments, the large exogenous genomic DNA/central region from the first organism is a homologous sequence of the corresponding central region of the second organism.

In certain embodiments, the large exogenous genomic DNA/central region from the first organism does not share significant sequence homology with the corresponding central region of the second organism, and thus merely corresponds to the corresponding central region of the second organism, by virtue of the fact that they are both flanked by the proximal region and the distal region for the purpose of homologous recombination.

Exemplary sizes of the homologous or corresponding central region of the second organism are: 10 kb, 15 kb, 20 kb, 25 kb, 30 kb, 35 kb, 40 kb, 45 kb, 50 kb, 60 kb, 80 kb, 100 kb, 150 kb, 200 kb, 250 kb, 300 kb, or 350 kb.

In certain embodiments, the length of the proximal region and the length of the distal region both are sufficiently long to support homologous recombination.

Exemplary sizes of the proximal region are: 10 kb, 15 kb, 20 kb, 25 kb, 30 kb, 35 kb, 40 kb, 45 kb, or at least about 50 kb. Exemplary sizes of the distal region are: 10 kb, 15 kb, 20 kb, 25 kb, 30 kb, 35 kb, 40 kb, 45 kb, or at least about 50 kb.

In certain embodiments, the homologous or corresponding central region is about 25-30 kb, preferably the central region is about 20 kb. Preferably, the proximal region and the distal region are each about 10 kb.

In certain embodiments, the size of the central region, the homologous central region, the proximal region, and the distal region, are independently selected from a range, the lower and higher ends of the range are defined by any of the value recited above, such as 20-300 kb for the central region, 15-250 kb for the homologous central region, 5-25 kb for the proximal and distal regions, etc.

In certain embodiments, the first organism and the second organism are the same species. The first organism and the second organism may be different strains of mouse or rats (e.g., inserting a gene from the C57BL/6J mouse strain into the FVB/NJ mouse strain).

In certain embodiments, the first organism and the second organism are different species. For example, the first organism can be human, and the second organism can be a rodent, such as a mouse or a rat.

In certain embodiments, the first and the second organisms are independently selected from: a human, a primate, a non-human primate, a mammal, a non-human mammal, a rodent (such as a mouse, a rat, a hamster, a guinea pig, a rabbit), a livestock animal (such as a cattle, a pig, a horse, a sheep, a goat, a camel, a llama), a pet (such as a cat or a dog), a fish (e.g., zebra fish), a frog, an insect, or a bacterium.

In certain embodiments, the first organism is human, and the second organism is mouse or rat.

In certain embodiments, the artificial genomic DNA is useful for homologous recombination in ES cells, which may require the use of selection markers. Thus in such embodiment, the artificial genomic DNA may have the following characteristics: (1) the central region comprises a first selectable marker cassette at the proximal end of the central region, and/or a second selectable marker cassette at the distal end of the central region, wherein: (a) the first selectable marker cassette comprises a first selectable marker (e.g., Neo^{R}) flanked by a pair of first recognition sites (e.g., FRT) compatible with a first site-specific recombinase (e.g., Flp), and, (b) the second selectable marker cassette comprises a second selectable marker (e.g., Bsd^{R}) flanked by a pair of second recognition sites (e.g., attB/attP) compatible with a second site-specific recombinase (e.g., φC31), and, (2) the central region comprises a deletable region (e.g., segments GH and KL) flanked by a pair of third recognition sites (e.g., loxP) compatible with a third site-specific recombinase (e.g., Cre).

Suitable site-specific recombinases that can be used as the first-, second-, and/or third-site-specific recombinases include: Tyr recombinases such as Cre, Dre, Flp, KD, B2 and B3; Tyr integrases such as λ, HK022, and HP1; Ser resolvase/invertases such as γδ, ParA, Tn3, and Gin; and Ser integrases such as φC31, Bxb1, and R4.

In certain embodiments, the deletable region is adjacent and distal to the first selectable marker cassette, and wherein one of the pair of third recognition sites is at the proximal end of the first selectable marker cassette.

In certain embodiments, the first selectable marker is Neo^{R} flanked by FRT. In certain embodiments, the second selectable marker is Bsd^{R} or Puro^{R} flanked by attB/attP. In certain embodiments, the pair of third recognition sites are loxP.

A related aspect of the present invention provides a vector compatible for CRISPR/Cas9-generated double-stranded break (DSB)—homologous recombination (HR)-mediated knock-in in a zygote, the vector comprising any of the genomic DNA described herein, optionally with the proviso that the first selectable marker and/or the second selectable marker, when present, are removed by the first and second site-specific recombinases, respectively.

Another related aspect of the invention provides a vector compatible for homologous recombination in embryonic stem (ES) cells, the vector may comprise any of the artificial genomic DNA described herein.

In accordance with the present invention, homologous recombination is facilitated by double strand breaks (DSBs) created by endonucleases. In certain embodiments, the endonuclease comprises CRISPR/Cas9 and one or more single guide RNA(s) (“sgRNA” or “gRNA” for short). In certain embodiments, the enzyme can be introduced by introducing vector(s) or coding sequence encoding the CRISPR/Cas9, and one or more sgRNA(s). In certain embodiments, the vector or coding sequence encoding the CRISPR/Cas9 is a CRISPR/Cas9 mRNA.

In certain embodiments, isolated Cas9 protein can be introduced into the cell (e.g., a zygote or an ES cell, through microinjection or electroporation) directly. The Cas9 protein may be in the form of a Cas9 riboprotein, which is a Cas9 protein/gRNA complex. Or the Cas9 protein may be without any gRNA, such that the Cas9 protein and the one or more gRNAs are co-introduced into the zygote or ES cell to allow the formation of the Cas9 protein/gRNA complex in situ inside the cell.

In certain embodiments, the CRISPR/Cas system comprises wild-type Cas9. For purposes of this application, Cas9 protein is not limited to the wild-type (wt) Cas9 found in Streptococcus pyogenes. It is intended to cover amino acids 7-166 or 731-1003 of the Cas9/Csnl amino acid sequence (of Streptococcus pyogenes), as depicted in FIG. 3 and SEQ ID NO: 8 of WO 2013/176772 (incorporated by reference); the corresponding portions in any one of the amino acid sequences SEQ ID NOs: 1-256 and 795-1346 of WO 2013/176772 (incorporated by reference); and the corresponding portions in any one of the amino acid sequences of the orthogonal Cas9 sequences from S. pyogenes, N. meningitidis, S. thermophilus and T. denticola (see, Esvelt et al., Nature Methods, 10(11): 1116-1121, 2013, incorporated by reference).

Other suitable endonucleases that can be used in the present invention can be an endonuclease that cuts the genome at a specific site, including Zinc finger nuclease (ZFN), a Transcription Activator-Like Effector Nuclease (TALEN), CRISPR/cpf1, or a meganuclease (such as an engineered meganuclease re-engineered homing endonuclease), or a combination thereof. For example, a DSB close to the junction of the proximal region and the homologous central region can be created by ZFN, and a DSB close to the junction of the distal region and the homologous central region can be created by CRISPR/Cas.

Preferably, the cleavage and/or recognition sites of the ZFN, TALEN, CRISPR/cpf1, or meganuclease are within a short distance from the junction of the proximal region and the homologous central region, or from the junction of the distal region and the homologous central region. For example, the short distance can be within 250 bp, 200 bp, 150 bp, 100 bp, 80 bp, 50 bp, 40 bp, 30 bp, 20 bp, 10 bp, or 5 bp of either of the junctions. In certain embodiments, the cleavage and/or recognition sites are within the homologous central region to be deleted.

In order to function as an endonuclease for use in the methods of the invention, Cas9 protein is required to form a functional complex with a gRNA.

According to a preferred embodiment of the invention, four specific gRNAs (i.e., two pairs) are used in the methods of the invention, each targeting a specific Cas9 cleavage site around the endogenous genomic DNA to be replaced by the large exogenous genomic DNA. That is, two guide RNAs (i.e., one pair) target the proximal end of the endogenous genomic DNA sequences to be deleted, and two guide RNAs (i.e., one pair) target the distal end of the endogenous genomic DNA sequences to be deleted.

While not wishing to be bound by any particular theory, such redundancy of four specific guide RNAs is advantageous for the insertion of large exogenous genomic DNA. One plausible explanation is that the combined activities of the two pairs of guide RNAs lead to more efficient double strand break (DSB) creation or more durable DSB persistence. Either more efficient double strand break (DSB) creation, or more durable DSB persistence, or both, improve the insertion of large exogenous genomic DNA by homologous recombination.

Thus in an exemplary embodiment of the present invention, two pairs of gRNAs are provided, comprising: (1) a first pair of sgRNAs (i.e., the first gRNA and the second gRNA) that directs the CRISPR/Cas9 to create a first double-stranded break (DSB) at or near the proximal end of the endogenous genomic DNA; and, (2) a second pair of sgRNAs (i.e., the third gRNA and the fourth gRNA) that directs the CRISPR/Cas9 to create a second double-stranded break (DSB) at or near the distal end of the endogenous genomic DNA.

In other embodiments, however, only one of the first pair of gRNAs, e.g., the first gRNA or the second gRNA, is used to direct the CRISPR/Cas9 to create a first double-stranded break (DSB) at or near the proximal end of the endogenous genomic DNA.

In related embodiments, only one of the second pair of gRNAs, e.g., the third gRNA or the fourth gRNA, is used to direct the CRISPR/Cas9 to create a second double-stranded break (DSB) at or near the distal end of the endogenous genomic DNA.

That is, in certain embodiments, one gRNA is used to create the first DSB at or near the proximal end of the endogenous genomic DNA, while two gRNAs are used to create the second DSB at or near the distal end of the endogenous genomic DNA. In certain other embodiments, two gRNAs are used to create the first DSB at or near the proximal end of the endogenous genomic DNA, while one gRNA is used to create the second DSB at or near the distal end of the endogenous genomic DNA. In yet other embodiments, one gRNA is used to create the first DSB at or near the proximal end of the endogenous genomic DNA, and one gRNA is used to create the second DSB at or near the distal end of the endogenous genomic DNA.

Preferably, independent of the number of gRNAs used to create the DSBs, in certain embodiments, each of the gRNA is independently selected based on their proximity to the proximal junction or the distal junction. That is, the first gRNA and the second gRNA can both be selected based on their proximity to the proximal junction, and the third gRNA and the fourth gRNA are both selected based on their proximity to the distal junction. As a result, the first DSB generated by Cas9/first gRNA and Cas9/second gRNA is closest to the proximal junction, and the second DSB generated by Cas9/third gRNA and Cas9/fourth gRNA is closest to the distal junction.

Independent of the number of gRNAs used to create the DSBs, in certain other embodiments, the gRNAs are independently selected not based on their proximity to the proximal or distal junctions, but are selected based on their predicted quality, as measured by scores generated by gRNA design algorithms, such as the standard algorithm available at http://crispr.mit.edu.

The selection and design of gRNA can be performed using well-known principles or online tools, based on user input such as target genome and sequence type. In general, the gRNA is a short synthetic RNA composed of a “scaffold” sequence necessary for Cas9-binding and a user-defined ˜20 nucleotide “spacer” or “targeting” sequence which defines the genomic target to be bound or modified by the targeting sequence. For simplicity, “gRNA targets a Cas9 cleavage site” refers to the fact that the spacer or targeting sequence of the gRNA is designed to bind to a genomic target sequence and cleave it at the cleavage site.

Preferably, the targeting sequence is sufficiently unique such that in theory it binds to a unique (compared to the rest of the genome) genomic target sequence. The target should be present immediately upstream (or 5′) of a Protospacer Adjacent Motif (or “PAM” sequence). The PAM sequence is absolutely necessary for target binding and the exact sequence is dependent upon the species of Cas9. In the most widely used Streptococcus pyogenes Cas9, the PAM sequence is 5′-NGG-3′ (“N” denotes any of the 4 standard nucleotides). Other PAM sequences for additional Cas9 in different species are known in the art. See exemplary PAM sequences listed below.

| Species/Variant of Cas9 | PAM Sequence |
|---|---|
| Streptococcus pyogenes (SP); SpCas9 | NGG |
| SpCas9 D1135E variant | NGG (reduced NAG binding) |
| SpCas9 VRER variant | NGCG |
| SpCas9 EQR variant | NGAG |
| SpCas9 VQR variant | NGAN or NGNG |
| Staphylococcus aureus (SA); SaCas9 | NNGRRT or NNGRR(N) |
| Neisseria meningitidis (NM) | NNNNGATT |
| Streptococcus thermophilus (ST) | NNAGAAW |
| Treponema denticola (TD) | NAAAAC |

The Cas9-gRNA complex will bind any target genomic sequence with a PAM, but Cas9 only cleaves the target genomic sequence if sufficient homology exists between the gRNA spacer and target genomic sequence. The end result of Cas9-mediated DNA cleavage is a double strand break (DSB) within the target genomic sequence, at a cleavage site that is about 3-4 nucleotides upstream of the PAM sequence.

In certain embodiments, the first gRNA and the second gRNA bind to different strands of the endogenous genomic DNA.

In certain embodiments, the third gRNA and the fourth gRNA bind to different strands of the endogenous genomic DNA.

In certain embodiments, the first gRNA and the second gRNA bind to the same strand of the endogenous genomic DNA.

In certain embodiments, the third gRNA and the fourth gRNA bind to the same strand of the endogenous genomic DNA.

In certain embodiments, the cleavage site of any selected gRNA is within about 250 bp from a proximal junction (for the first and second gRNAs) or a distal junction (for the third and fourth gRNA). Preferably, the cleavage site is within about 100 bp, 50 bp, or 10 bp from the proximal junction for the first and second gRNAs, and within about 100 bp, 50 bp, or 10 bp from the distal junction for the third and fourth gRNAs.

Introduction of the BAC-vector carrying the large exogenous genomic DNA to be delivered to the target cell may be effected by any method known to those of skill in the art.

In certain embodiments, the vector carrying the large exogenous genomic DNA, the Cas9 protein or coding sequence, and one or more sgRNA(s), are introduced into the zygote through microinjection or electroporation.

In certain embodiments, foreign genes on the large exogenous genomic DNA can be delivered by transfection or electroporation into ES cells, followed by selection using selection markers.

Microinjection is a well-known technique used to introduce foreign substance (e.g., DNA, RNA, and/or protein) into certain cells (such as zygotes) or early stage embryos. In certain embodiments, a sufficient amount of the BAC vector carrying the large exogenous genomic DNA, along with the Cas9 protein or coding sequence, and one or more sgRNA(s), are microinjected into the zygote.

The viscosity of the injected solution containing the BAC-vector and large exogenous DNA is found to be essential for the success homologous recombination to proceed. The viscosity of the injection solution relates to the amount of the donor BAC-vector containing the large exogenous DNA. Preferably, microinjection is performed using optimal viscosity of about 1-10 ng/μL of the BAC containing the large exogenous genomic DNA, more preferably, 2-8 ng/μL, and most preferably, about 5 ng/μL.

Electroporation of CRISPR/Cas9 components (Cas9 coding sequence and gRNAs) and donor DNA (BAC carrying the large exogenous genomic DNA) can be carried out according to the method described in WO 2016/054032 (incorporated herein by reference).

In certain embodiments, the method further comprises transferring the ES cell or the zygote into a pseudo-pregnant female.

In mice, pseudopregnant females are readied by mating six- to eight-week-old female mice in natural estrus with vasectomized males.

Zygotes processed for same day transfer to pseudopregnant females can be removed from culture and placed into a pre-warmed suitable medium (such as M2 medium) and transferred via the oviduct into 0.5 days post coitum pseudopregnant females (age 9-11 wks).

Once the large exogenous genomic DNA is inserted into a host mammal using the methods of the invention, correct genomic insertion can be verified in the resulting transgenic animal (e.g., mouse) or progeny thereof.

Such verification typically includes one or more of genotyping animals that potentially carry the transgene, polymerase chain reaction amplification of junctional sequences, direct sequencing of certain stretches of genomic DNA (such as DNA junction sequence where the transgene is inserted into the host genome), and genetic mapping to determine the insertion location with respect to known genetic markers in the host genome. Such techniques are well-known in the art.

### EXAMPLES

The present invention is further illustrated by the following Examples. These Examples are provided to aid in the understanding of the invention, and are not to be construed as a limitation thereof.

### Example 1 Knock-in a 25-Kilobase Pair BAC-Derived Genomic DNA by CRISPR/Cas9-Stimulated Homologous Recombination

This example describes the use of the invention described herein in humanizing specific regions of the mouse genome using large exogenous genomic DNA from human-genomic DNA segments with extents of 10's to 100's of kilobase pairs (kb). More specifically, this example provides a CRISPR-driven replacement (e.g., humanization) of an approximately 17-kilobase pair (kb) segment of a mouse tumor suppressor gene Bcl2l11 with an orthologous, disease-associated, 25-kb segment of the corresponding human gene BCL2L11.

a) A Large Exogenous Genomic DNA from Human

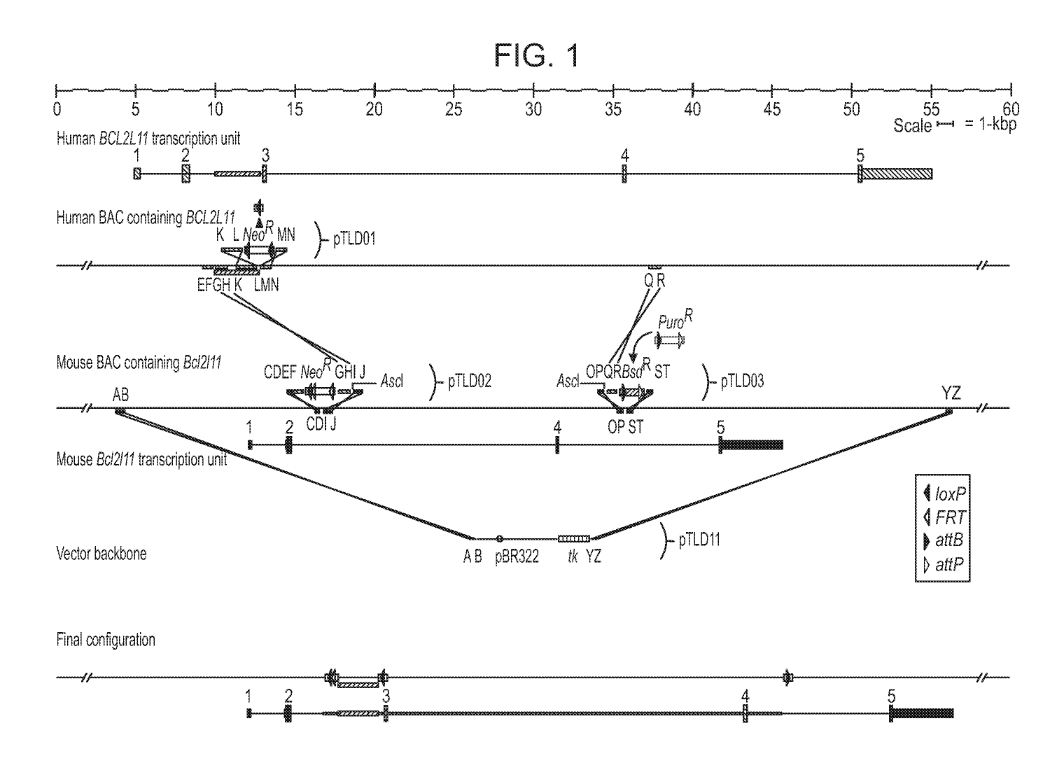

BAC DNAs were purified from a BAC clone containing the gene of interest, e.g., the human BCL2L11 gene (human: library RP11, clone 695-B-23) in this case. Purified DNA was then electroporated into the recombinogenic E. coli strain, SW102. See FIG. 1, third line from the top, showing a human BAC containing the human BCL2L11 gene.

b) Preparation of a Targeting Vector Using a Bacterial Artificial Chromosome (BAC)

BAC DNA was purified from a BAC clone containing the target genomic locus, e.g., the corresponding mouse Bcl2l11 gene (mouse: library RP23, clone 331-K-22) in this case. Purified DNA was then electroporated into the recombinogenic E. coli strain, SW102.

Segments from the mouse and human BACs were amplified using the oligonucleotides described in Table 1 below.

The amplified genomic DNA segments from the mouse and human BACs were then restriction-digested at sites incorporated into the oligonucleotides (see Table 1), gel-purified, and assembled into small plasmid vectors as follows:

Segments KL and MN were cloned along with the neomycin resistance gene-(Neo^{R}-) containing EcoRI/BamHI fragment of PL452, into a pBluescript II vector (Agilent Technologies, Santa Clara, Calif. USA) modified to contain an R6Kγ origin of replication. This plasmid is named pTLD01.

Segments CD, EF, GH, and IJ were cloned along with the neomycin resistance gene-(Neo^{R}-) containing EcoRI/BamHI fragment of PL451, into a pBluescript II (Agilent Technologies, Santa Clara, Calif. USA) vector modified to contain an R6Kγ origin of replication. This plasmid is named pTLD02.

Segments OP, QR, and ST were cloned along with the blasticidin resistance gene-(Bsd^{R}-) containing EcoRI/BamHI fragment of pTLD08 (a PL452 derivative carrying attB, attP, and Bsd^{R}), into a pBluescript II vector (Agilent Technologies, Santa Clara, Calif. USA) modified to contain an R6Kγ origin of replication. This plasmid is named pTLD03.

For larger insert genomic DNA, the BAC can be used directly for the method described herein. However, in this case, since the full capacity of the BAC vector was not required, we used a reduced size (from 225 kbp to 70 kbp) version of the vector having an alternative vector (i.e., pBR322) backbone.

Specifically, to reduce the size of the construct, segments AB and YZ were cloned into a pBR322-based vector along with the negatively selectable thymidine kinase (tk) gene. This plasmid is named pTLD11.

The pTLD11 vector was then used according to the following steps:

First, the pTLD01 plasmid was used with standard recombineering approaches to place a loxP-flanked neomycin resistance cassette (Neo^{R}) just distal to the 2,903-bp deletion region in the human BCL2L11-containing BAC. After transferring the BAC containing the modified human BCL2L11 gene to the Cre-expressing E. coli strain, SW106, the Neo cassette was removed by exposing cells to arabinose, leaving a single loxP site remaining. See FIG. 1, third line from the top, and the structure of the pTLD01 plasmid, showing the homologous recombination scheme using the human KL and MN homology arms. Also see the 2^{nd} to the last line of FIG. 1, showing the remaining single loxP site.

Next, plasmid pTLD02 was used with standard recombineering techniques to place the EF segment of human DNA, a loxP site, an FRT-flanked Neo cassette, and the GH segment of human DNA just distal to mouse Exon 2 in the mouse Bcl2l11-containing BAC. See the fourth line of FIG. 1, left side, and the structure of the pTLD02 plasmid, showing the homologous recombination scheme using the mouse CD and IJ homology arms.

Next, plasmid pTLD03 was used with standard recombineering techniques to place the QR segment of human DNA, and an attB/attP-flanked blasticidin resistance (Bsd^{R}) cassette, slightly distal to mouse Exon 4 in the BAC containing the pTLD02-modified mouse Bcl2l11 genomic DNA described above. See the fourth line of FIG. 1, right side, and the structure of the pTLD03 plasmid, showing the homologous recombination scheme using the mouse OP and ST homology arms.

Next, plasmid pTLD11 was linearized with HindIII and used with standard recombineering procedures to retrieve the AB to YZ (AZ) segment of the mouse Bcl2l11 gene from the BAC containing the pTLD02/pTLD03-modified mouse Bcl2l11 genomic DNA described above, becoming pTLD14. See the fourth and the sixth lines (i.e., the structure of the pTLD11 plasmid) of FIG. 1, showing the homologous recombination scheme using the mouse AB and YZ homology arms.

The resulting vector, pTLD14, (see FIG. 1, the 6^{th} line), contains the entire mouse Bcl2l11 gene, as well as the pTLD02 insertion fragments (i.e., EF segment of human DNA, a loxP site, an FRT-flanked Neo cassette, and the GH segment of human DNA), and the pTLD03 insertion fragments (the QR segment of human DNA, and an attB/attP-flanked blasticidin resistance (Bsd^{R}) cassette).

c) Insertion of a Large Exogenous Genomic DNA into the Targeting Vector

To begin the assembly of our humanized donor vector, the plasmid pTLD14 was purified, digested with AscI, and its two major fragments resolved by agarose gel electrophoresis. The smaller of the two linear fragments, defined by the AscI cutting sites and the mouse IL and OP genomic DNA fragments, was discarded.

The larger of the two linear fragments was gel-purified and electroporated into recombinogenic E. coli cells containing the loxP-modified human BAC clone described above, thus capturing the 27,282-bp human segment between flanking mouse homology arms, becoming plasmid pTLD15 (See FIG. 1, lines 3 and 4).

For the purpose of CRISPR/Cas9-based zygotic microinjection, the final Neo^{R}/Bsd^{R}-containing vector (plasmid pTLD15) was electroporated; first, into the FLP-expressing E. coli strain SW105 to remove Neo^{R} (making plasmid pTLD66), and next, into a ϕC31 recombinase-expressing E. coli strain (an SW105 derivative) to remove Bsd^{R}. The final vector was named pTLD67, which contains the 27-kb exogenous human genomic DNA flanked by two mouse homology arms. See the last two lines in FIG. 1.

The resulting targeting vector (pTLD67)/donor molecule contained a 27,282-bp central segment of the human BCL2L11 gene flanked by 12,773- and 26,632-bp homology arms consisting of the proximal and distal regions of the mouse Bcl2l11 gene, respectively.

There are at least three features in the resulting targeting vector (pTLD67)/donor molecule: 1) an 18 kb central segment of the mouse Bcl2l11 gene that was replaced/humanized by a large exogenous genomic DNA from the homologous human gene BCL2L11; 2) selectable markers were initially placed immediately 5′ and 3′ of the humanized segment, but such selectable markers were removed in the final pTLD67 vector for our CRISPR/Cas9-based experiment (in contrast, such selection markers were retained for the ES-cell based traditional approach, see comparative example below); and 3) a 2,903-bp region within one of the humanized introns was flanked with loxP sites, in order to model a disease-associated deletion observed in 12% of the East Asian population.

As a general approach, however, the first feature is not limited to replacing endogenous genomic DNA with exogenous genomic DNA that shares sequence homology. Meanwhile, the third feature is generally not required, but can be useful for certain specific uses.

d) Preparation of CRISPR/Cas9 Guide RNAs (gRNAs)

All single-guide RNAs (sgRNAs or gRNAs) were designed using a standard approach, such as the algorithm available at http://crispr.mit.edu. These sgRNAs, shown in Table 2, were designed along two concepts.

In the first, the two highest scoring sgRNAs (one in each orientation) within a 250-bp region were selected from both the 5′ and 3′ ends of the 17-kbp segment of the mouse Bcl2l11 segment being replaced. In the second, two internal sgRNAs (one in each orientation) closest to each end of the replaced segment were selected regardless of their overall score. Guides were produced according to the method of Briner, et al. (Molecular cell. 56(2):333-339, 2014, PubMed PMID: 25373540, incorporated herein by reference). Cas9 mRNA (CRISPR associated protein 9 mRNA, 5-methylcytidine, pseudouridine) was purchased from TriLink Biotechnologies (San Diego, Calif.).

Overall, four guide RNAs were designed at each end of the mouse Bcl2l11 gene segment to be replaced including two (one in each orientation) with the top design score, and two (one in each orientation) located closest to the outermost ends.

e) Cas9 Coding Sequence

CRISPR Associated Protein 9 mRNA/5-methylcytidine/pseudouridine (Cas9 mRNA/5meC/Ψ) from Streptococcus pyogenes (strain SF370) was obtained from TriLink Biotechnologies (San Diego, Calif.) and used in our microinjection mixes at a final concentration of about 100 ng/μL.

f) Microinjection of the Targeting Vector, CRISPR/Cas9 gRNAs, and Cas9 Coding Sequence

Although other means such as electroporation may also be used, we used microinjection to introduce the targeting vector containing the large exogenous human genomic DNA and the CRISPR/Cas9-gRNAs into mouse zygotes.

To prepare mouse zygote for microinjection, C57BL6/J donor female mice (age 3 weeks) were superovulated to maximize embryo yield. Each donor female received 5 international unit (IU) intraperitoneally (IP) of Pregnant Mare Serum Gonadotropin (PMSG) (Prospect HOR-272) followed 47 hours later by 5 IU (IP) of human chorionic gonadotropin (hCG) (Prospec HOR-272) Immediately post administration of hCG the females were mated 1:1 with C57BL6/J stud males and 22 hours later checked for the presence of a copulation plug. Females displaying a copulation plug were euthanized and the oviducts excised and placed into M2 media.

Prior to clutch collection the oviducts were placed in M2 media containing hyaluronidase (Sigma H3506) (0.3 mg/mL). The oocyte clutch was removed by mechanically lysing the ampulla and the clutches were allowed to incubate in the M2 containing hyaluronidase until the cumulus mass had broken down enough to expose the oocytes/prospective zygotes. The oocytes/prospective zygotes were transferred through several washes of fresh M2 and then (through the process of visual grading) individual identified zygotes were separated and transferred to microdrops of K-RCVL (COOK K-RVCL) medium that had been equilibrated under mineral oil (SigmaM8410) for 24 hours in a COOK MINC benchtop incubator (37° C., 5% CO₂/5% O₂/Nitrogen).

Microinjection mixes were prepared as shown in Table 3. Approximately 80 C57BL/6NJ zygotes were microinjected (in one to two technical replicates with each microinjection mix described above). Microinjection mixes contained four guides (either those with the highest scores or those with the most terminal positions within the mouse Bcl2l11 segment to be replaced) and varying concentrations of donor DNA (1, 5, or 10 ng/μL).

Specifically, zygotes were removed from culture and placed onto a slide containing 150 μL of fresh M2 medium. Microinjection occurred on a Zeiss AxioObserver.D1 using Eppendorf NK2 micromanipulators in conjunction with Narashige IM-5A injectors. Standard zygote microinjection procedure was followed with special care made to deposit material into both the pronucleus and the cytoplasm of the subject zygote. Needles for microinjection were pulled fresh daily using WPI TW100F-4 capillary glass and a Sutter P97 horizontal puller. Injected zygotes were removed from the slide and rinsed through three 30 μL drops of equilibrated K-RCVL before being placed into a separate 30 μL microdrop of equilibrated K-RCVL where they were subsequently processed for embryo transfer (via the oviduct) on the day of injection.

g) Animal Husbandry

All mice were obtained from The Jackson Laboratory (Bar Harbor, Me.), housed on a bedding of white pine shavings, and fed NIH-31 5K52 (6% fat) diet and acidified water (pH 2.5 to 3.0), ad libitum. All experiments were performed with the approval of The Jackson Laboratory Institutional Animal Care and Use Committee (IACUC) and in compliance with the Guide for the Care and Use of Laboratory Animals (8th edition) and all applicable laws and regulations.

h) Preparation of a Pseudopregnant Female

Pseudopregnant females were readied by mating six- to eight-week-old female mice in natural estrus with vasectomized males.

Zygotes processed for same day transfer to pseudopregnant females were removed from culture and placed in a 1.8 mL screw-top tube (Thermo Scientific 363401) containing 900 μL of pre-warmed M2 medium for transport to the surgical station. The zygotes were removed from the tube and placed into culture (K-RCVL under oil-COOK MINC benchtop incubator 37° C., 5% CO₂/5% O₂/Nitrogen). At the time of transfer, the zygotes were removed from culture and placed into pre-warmed M2 medium and transferred via the oviduct into 0.5 days post coitum pseudopregnant CBYB6F1/J females (age 9-11 wks).

Pregnancies proceeded to term and pups were delivered naturally.

i) Implantation of Microinjected Zygotes into Pseudopregnant Females

Mouse zygotes microinjected above were transferred to pseudopregnant females by standard techniques, and were allowed to go to term, where they were reared by the dams until weaning at four weeks of age.

j) Verification of Correct Genomic Insertion—Genotyping

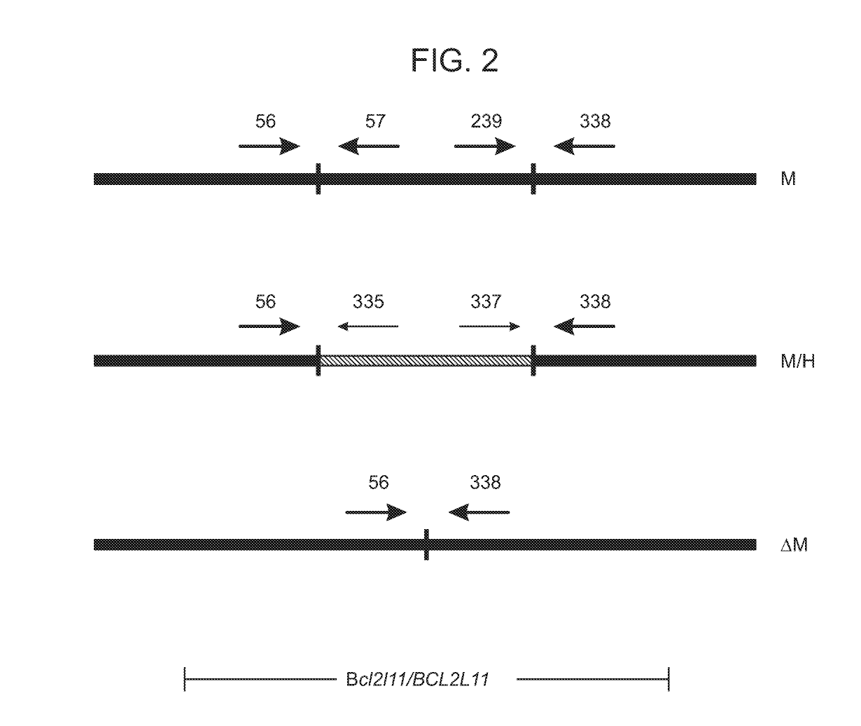

Potentially chimeric mice, arising from the microinjection of 1-celled zygotes (CRISPR approach), and their progeny were genotyped at designed Cas9 binding sites using the oligonucleotide primers described in Table 4. As shown in FIG. 2, these primers were used in pairs, in separate PCR reactions designed to amplify DNA across: 1) the Cas9 binding sites of intact (or small INDEL-containing) mouse alleles, 2) the mouse/human junctions of humanized alleles (or randomly integrating transgenes), and 3) the breakpoints of any deletion-bearing alleles.

k) Verification of Correct Genomic Insertion—Sanger Sequencing

For more detailed analysis of specific alleles, PCR products from genotyping reactions were purified and sequenced by JAX Scientific Services according to the method developed by Sanger. PCR products were purified using HighPrep PCR magnetic beads (MagBio Genomics, Gaithersburg, Md. USA). Cycle sequencing was performed using a BigDye Terminator Cycle Sequencing Kit, version 3.1 (Applied Biosystems, Foster City, Calif. USA).

Sequencing reactions contained 5 μL of purified PCR product (3-20 ng) and 1 μL of primer at a concentration of 5 pmol/μL. Sequencing reaction products were purified using HighPrep DTR (MagBio Genomics, Gaithersburg, Md. USA). Purified reactions were run on an Applied Biosystems 3730×1 DNA Analyzer (Applied Biosystems, Foster City, Calif. USA).

Sequence data were analyzed using Sequencing Analysis Software, version 5.2 (Applied Biosystems, Foster City, Calif. USA). Resulting sequence (.abi) files were imported into Sequencher, version 5.0.1 (Gene Codes Corporation, Ann Arbor, Mich. USA), for further analysis.

l) Verification of Correct Insertion Locus—Genetic Mapping

To show that the human segment of BCL2L11 had indeed replaced its mouse counterpart in the orthologous Bcl2l11 locus, we used genetic mapping to localize the humanized segment of the BCL2L11/Bcl2l11 gene (FIGS. 3A and 3B).

Two backcrosses were established using the following approach. First, FVB/NJ females were crossed to CS7BL/6NJ males carrying the humanized segment to obtain F₁ hybrid (FVBB6NF1/J) progeny. These progeny were then genotyped for the presence of the humanized segment. Males carrying the human sequence (FVBB6NF1/J-BCL2L11) were backcrossed to either FVB/NJ females or CS7BL/6NJ females to generate N₂ progeny.

These backcross schemes can be annotated as follows:
CS7BL/6NJ×FVBB6NF1/J-BCL2L11
FVB/NJ×FVBB6NF11J-BCL2L11

N₂ progeny from each backcross (along with appropriate controls) were genotyped using KASP-chemistry (LGC Limited, Teddington, UK) across a set of approximately 150 single-nucleotide polymorphism (SNP) markers distributed roughly equally across the mouse genome. Concordance between each marker in the set and the humanized segment was calculated by chi-square (χ²) analysis.

### Results

1) Successful Gene Targeting

At term, a total of 94 pups were born; six were stillborn and six did not survive to four weeks of age. Eighty-two mice were weaned and distributed among experiments as shown in Table 4.

Both Experiment 3 (highest scoring guides, 5 ng/μL donor DNA) and Experiment 5 (guides closest to ends, 10 ng/μL donor DNA), shown in Table 3, resulted in no viable pups remaining at wean-age. Despite these results, Experiment 7 (conducted with a donor DNA concentration equal to that of Experiment 5, i.e., 10 ng/μL, see Table 3) and Experiment 8 (a replicate of Experiment 3, Table 3) resulted in seven and 21 pups, respectively, suggesting that the lack of pups in Experiments 3 and 5 was due to technical failure rather than anything systematically wrong with the experimental design.

To genotype these 82 progeny PCR assays were designed to span each of the proximal and distal mouse/human junctions and to span the 17-kbp mouse region to be replaced. The results of these experiments are shown in Table 5. As noted, PCR assays designed to span each of the proximal and distal mouse/human junctions identified three founders that were positive for both (Experiment 2, guides closest to ends, 1 ng/μL donor DNA; Experiment 6, guides closest to ends, 5 ng/μL donor DNA; and Experiment 7, highest scoring guides, 10 ng/μL donor DNA, see Table 3). PCR assays designed to span the 17-kbp mouse region to be replaced identified two of the three founders described above (Experiment 6, guides closest to ends, 5 ng/μL donor DNA; and Experiment 7, highest scoring guides, 10 ng/μL donor DNA, Table 3).

2) Successful Germline Transmission

To further explore the inheritance of these genetic changes, we mated the human insertion/deletion-positive P₀s from Experiments 2, 6, and 7 to C57BL/6J mice and genotyped their progeny. The results of these analyses are shown in Table 5.

As shown, the human insertion-positive P₀ mouse (male) from Experiment 2 (guides closest to ends, 1 ng/μL donor DNA) failed to transmit the humanized allele to any of 29 of its N₁ progeny, suggesting that the P₀ mouse is mosaic with a germline consisting primarily of unmodified wildtype cells.

In contrast, the human insertion- and deletion-positive P₀ mouse (male) from Experiment 7 (highest scoring guides, 10 ng/μL donor DNA) transmitted its deletion-bearing allele to four of its 21 N₁ progeny. This P₀ mouse, however, did not transmit the human insertion-bearing allele to any of these 21 mice again, suggesting that the P₀ mouse is mosaic with a germline consisting of relatively few human insertion-bearing cells.

Interestingly, the human insertion- and deletion-positive P₀ mouse (female) from Experiment 6 (guides closest to ends, 5 ng/μL donor DNA) transmitted either a human insertion-bearing allele or a deletion-bearing allele to all of its 13 N₁ progeny, but never both, implying that this animal is breeding as a true heterozygote with a genotype of both human insertion- and deletion-bearing alleles at the Bcl2l11 locus.

Subsequent breeding of three select N₁ mice (two bearing the human insertion and one bearing the deletion) gave results consistent with Mendelian expectations. Mating males with B6N.Cg-Tg(Sox2-Cre)1Amc/J female mice resulted in progeny in which the loxP-flanked 2.9-kbp human intronic segment was deleted, as designed.

3) Concentration of Donor DNA (Exogenous DNA in Targeting Vector) and Guide RNA Position Affect Efficiency

Among the experiments in which donor DNA was detected in P₀ mice (Experiments 2, 6, and 7 of Table 3), DNA donor concentrations of 1, 5, and 10 ng/μL were represented but the resulting mice show varying degrees of mosaicism. In Experiment 2, where donor DNA concentration was at its lowest (1 ng/μL), donor DNA was not detected among N₁ progeny (0/50) suggesting that integration of the donor DNA occurred at multicellular stage of embryonic development and that those cells that did acquire the donor DNA did not contribute to the germline at an appreciable level.

In Experiment 6 of Table 3, where donor DNA concentration was at an intermediate level (5 ng/μL), donor DNA was detected among nearly half of all N₁ progeny (14/31) suggesting that integration of the donor DNA occurred at the one-cell (zygotic) stage of embryonic development, that that cell gave rise to all cells of the germline, and that the donor DNA was passed, during meiosis, into half of the population of mature spermatozoa. This result is consistent with our hypothesis that a deletion, of the 17-kbp mouse segment to be replaced, occurred at the Bcl2l11 locus in the homologous chromosome in the zygote, and was transmitted, in repulsion to the DNA insertion, to all remaining progeny (17/17). This result is entirely congruent with the optimal desired outcome, i.e., where the P₀ zygote undergoes biallelic modification, develops into a mouse with no mosaicism, and transmits one or the other variant alleles in equal numbers (50%:50%) to the population of mature spermatozoa.

In Experiment 7 of Table 3, where donor DNA concentration was at the highest level tested (10 ng/μL), the 17-kbp deletion was detected in only 25% of all N₁ progeny (14/56), and the donor DNA, present in the P₀ mouse, was not transmitted to the N₁ generation at all (0/56). These results can be explained assuming a scenario whereby a deletion occurred in one Bcl2l11 allele, in a single blastomere, at or near the two-cell stage, and that this deletion-bearing cell gave rise to roughly half of the developing premeiotic germline and a fourth of all mature (postmeiotic) germ cells. At some later point in blastogenesis, one can hypothesize that an insertion of donor DNA occurred, but in so few cells as to not contribute to the germline in an appreciable way.

While not wishing to be bound by any particular theory, a number of aspects in Experiment 7 of Table 3 may have contributed to its less than optimal result.

First, due to its viscosity, a donor DNA preparation with a DNA concentration that is too high may not be efficiently delivered through the microinjection needle to the zygote, or delivered in a form that is less conducive to promoting Cas9 activity and/or HDR.

Second, the guides designed for this experiment, although designed to have an optimal score, did not have what we surmised to be an optimal position, near the ends of the mouse DNA segment to be replaced. It may be that, in experiments of this type, guide position represents a more significant design parameter than guide score alone. It is interesting to note that, among all experiments using guides designed for high score optimization, only in Experiment 7, where donor DNA concentration was at the highest level tested (10 ng/μL), was any evidence of donor DNA incorporation seen, and even here it was at a level apparently so low in the P₀ founder mouse as to not transmit the modified allele to N₁ mice. Recall that, in the previously mentioned Experiment 6, where an optimal result was achieved, donor DNA concentration was only 5 ng/μL. It is entirely possible that the successful result seen in that instance was driven by superiorly performing/positioned (nearest the end) guides even at what could prove to be a suboptimal donor DNA concentration. Comparing Experiment 6 with Experiment 7, it is interesting to note that the experiment with the higher donor DNA concentration (Experiment 7, 10 ng/μL) did achieve a higher rate of incorporation (as a percentage of live born mice, 14.3% versus 5.6%) but a lower quality of allele modification in the single founder recovered (mosaicism/transmission of only one modified allele at low frequency compared to nonmosaicism/transmission of both modified alleles at maximum frequency).

One may speculate that DNA concentration may be the most important parameter related to the introduction of DNA into individual zygotes; whereas, guide design may prove to be the most important factor for promoting more frequent deletion formation and more efficient HDR once donor DNA has entered the cell.

4) Integration of Exogenous DNA Occurred at the Designed Locus

We used an outcross-backcross genetic mapping strategy as a means of localizing the insertion site of BAC-derived human BCL2L11 sequences. Twenty-two N₂ progeny were analyzed from the C57BL/6NJ×(FVB/NJ×C57BL/6NJ) backcross and twenty-eight progeny from the FVB/NJ×(FVB/NJ×C57BL/6NJ) backcross. Analysis of the data demonstrates strong linkage between the human BCL2L11 segment and several genetic markers on mouse Chromosome 2 (See FIGS. 3A and 3B).

In the backcross to C57BL/6NJ, the marker with strongest linkage, marker rs13476756, had a log-odds ratio (LOD) of 6.58 (p<0.004). In the backcross to FVB/NJ, marker rs13476756 had a LOD score of 7.64 (p<0.0004).

Analysis of individual haplotypes (specifically, points of recombination in samples 261, 263, 266, 303, and 319) further narrows the insertion-critical region to a 45.2-Mbp region from marker rs4223406 (nucleotide 113,827,352) to marker rs3689600 (nucleotide 159,014,253) on Mouse Chromosome 2 (GRC38/mm10), which is consistent with integration into the 36,510-bp mouse Bcl2l11 gene that spans from nucleotide 128,126,038 to nucleotide 128,162,547.

Put another way, this analysis shows that both the mouse Bcl2l11 gene and the engineered human sequences must be co-localized within a region comprising less than 2% of the mouse genome. We conclude that integration of the human sequence has not occurred randomly, but has indeed occurred by homologous recombination as designed.

In sum, the above example shows that the described CRISPR/BAC technology can be used to introduce large exogenous DNA (such as a 25 kb human gene homologous to its mouse counterpart) in a directed fashion to the zygotic genome, and the resulting transgenic animal has the ability to pass the specifically targeted DNA through germline transmission to its progeny, as we have demonstrated by PCR, sequence, and linkage analysis.

### Example 2 Knock-In of a 25-Kilobase Pair BAC-Derived Donor Molecule by Traditional ES-Cell Based Homologous Recombination

In contrast to the CRISPR/Cas9 driven approach described in Example 1, we also used a traditional approach that involved classic ES cell targeting, dual selection, and recombinase-driven cassette removal.

The general steps in this traditional approach were substantially the same as those in Example 1, except for the following specific steps.

b′) Preparation of a Targeting Vector Using a Bacterial Artificial Chromosome (BAC)

Once the pTLD14 plasmid was obtained (see Example 1), we experienced some difficulty with blasticidin-based embryonic stem (ES) cell selection. Thus, we replaced the open reading frame (ORF) of Bsd^{R} with that of Puro^{R} through a negatively selectable rpsL intermediate.

The resulting vector, pTLD39, contained a 27,282-bp central segment of the human BCL2L11 gene flanked by 12,773- and 26,632-bp homology arms consisting of the proximal and distal regions of the mouse Bcl2l11 gene, respectively. Close to the 5′-end of the large exogenous human genomic DNA is a Neo^{R} cassette flanked by FRT sites. Close to the 3′-end of the large exogenous human genomic DNA is a Puro^{R} cassette flanked by attB and attP sites. The vector pTLD39 performed well in embryonic stem cells subjected to sequential neomycin/puromycin selections.

f′) Electroporation of ES Cell Targeting Vector pTLD39

We electroporated 25 μg of linear pTLD39 DNA into 1.5×10⁷ cells of the JM8-A3 (Strain: C57BL/6N) line of mouse embryonic stems cells. ES cells were then plated in ES+2i medium with sequential gentamycin (G418, 200 μg/mL, Gibco, Fisher Thermo Scientific, Waltham, Mass., USA) and puromycin (0.75 μg/mL, Sigma-Aldrich, St. Louis, Mo., USA) selection.

Surviving ES cell clones were propagated on ES+2i medium, karyotyped, further tested for the presence of the puromycin resistance cassette by PCR, and assessed for homology arm, insert, and neomycin resistance cassette count by quantitative PCR. Properly targeted clones were microinjected into 3.5-days post coitum (dpc) blastocysts (see below).

h′) Injection of Electroporated ES Cells to Blastocysts Before Implantation into Pseudopregnant Females

Properly targeted ES clones were microinjected into 3.5-dpc blastocysts, and the blastocysts were then transferred to pseudopregnant host dams, by standard techniques.

The resulting embryos were allowed to go to term; the pups were delivered naturally and reared by the dams until weaning at four weeks of age. The pups were then subjected to genotyping, Sanger sequencing, and genetic mapping, as in Example 1.

### Results

Following electroporation of the pTLD39 vector into the JM8-A3 line of ES cells and selection on G418, we assayed 89 surviving clones for the presence of the puromycin resistance cassette by PCR. Of these, twenty-seven (27) contained the puromycin cassette and were subjected to puromycin selection. Of these, four (4) clones survived and were assessed for homology arm, insert, and neomycin resistance cassette count by quantitative PCR. One (1) clone passed all of these tests for proper targeting of the central human BCL2L11 segment to the endogenous mouse Bcl2l11 gene.

ES cells from this clone were microinjected into blastocysts resulting in nine (9) high-quality chimeras. The four highest quality male chimeras were mated to C57BL/6NJ females resulting in two independent instances of germline transmission of the humanized allele. Although presumably identical, independent lines (genetic background: C57BL/6JN) were developed from each instance. Mating males with B6N.Cg-Tg(Sox2-Cre)1Amc/J female mice resulted in progeny in which the loxP-flanked 2.9-kbp human intronic segment was deleted, as designed.

Although the foregoing invention has been described in some detail by way of illustration and examples for purposes of clarity of understanding, this invention is not limited to the particular embodiments disclosed, but is intended to cover all changes and modifications that are within the spirit and scope of the invention as defined by the appended claims.

All publications and patents mentioned in this specification are indicative of the level of skill of those skilled in the art to which this invention pertains. All publications and patents are herein incorporated by reference to the same extent as if each individual publication or patent application were specifically and individually indicated to be incorporated by reference.

TABLE 1
Oligonucleotides Used in the Construction of Donor Vectors

| | | | | | | | | | | Pro- | Homol- | | | |
|---|---|---|---|---|---|---|---|---|---|---|---|---|---|---|
| Abbre- | Syn- | Orien- | | Genome | Chromosomal | | | | | duct | ogy | Overall | | |
| viation | onym | tation | Species | Build | Coordinates | | | | | Size | Length | Length | Sequence | Enzyme |
| A | oTLD38 | + | Mouse | mm10 | Chr | 2 | : | 128116776 | 128116795 | 424 | 20 | 31 | 5′-dCGCA | SpeI |
| | | | | | | | | | | | | | TACTAGTT | |
| | | | | | | | | | | | | | CCATCCGG | |
| | | | | | | | | | | | | | TCATTTCT | |
| | | | | | | | | | | | | | CTC-3′ | |
| B | oTLD39 | - | Mouse | mm10 | Chr | 2 | : | 128117158 | 128117177 | | 20 | 31 | 5′-dCGCA | HinDIII |
| | | | | | | | | | | | | | TAAGCTTT | |
| | | | | | | | | | | | | | TTTGCTTG | |
| | | | | | | | | | | | | | GTCCAGAT | |
| | | | | | | | | | | | | | TCC-3′ | |
| C | oTLD29 | + | Mouse | mm10 | Chr | 2 | : | 128129327 | 128129348 | 246 | 22 | 35 | 5′-dCGCA | NotI |
| | new | | | | | | | | | | | | TGCGGCCG | |
| | | | | | | | | | | | | | CATAGTTT | |
| | | | | | | | | | | | | | AATAACCA | |
| | | | | | | | | | | | | | CCAGGC | |
| | | | | | | | | | | | | | A-3′ | |
| D | oTLD30 | - | Mouse | mm10 | Chr | 2 | : | 128129528 | 128129548 | | 21 | 32 | 5′-dCGCA | HinDIII |
| | | | | | | | | | | | | | TAAGCTTA | |
| | | | | | | | | | | | | | ACTGACTG | |
| | | | | | | | | | | | | | TAGCCCCA | |
| | | | | | | | | | | | | | GAAA-3′ | |
| E | oTLD27 | + | Human | hg38 | Chr | 2 | : | 111124898 | 111124917 | 742 | 20 | 31 | 5′-dCGCA | HinDIII |
| | | | | | | | | | | | | | TAAGCTTT | |
| | | | | | | | | | | | | | ATTGCTCA | |
| | | | | | | | | | | | | | GAGGGTTT | |
| | | | | | | | | | | | | | GGA-3′ | |
| F | oTLD28 | - | Human | hg38 | Chr | 2 | : | 111125598 | 111125617 | | 20 | 31 | 5′-dCGCA | BamHI |
| | | | | | | | | | | | | | TGGATCCT | |
| | | | | | | | | | | | | | GATTTACC | |
| | | | | | | | | | | | | | TCACTGAA | |
| | | | | | | | | | | | | | GCC-3′ | |
| G | oTLD20 | + | Human | hg38 | Chr | 2 | : | 111125618 | 111125641 | 765 | 24 | 35 | 5′-dCGCA | EcoRI |
| | | | | | | | | | | | | | TGAATTCG | |
| | | | | | | | | | | | | | GCAGGCCT | |
| | | | | | | | | | | | | | TTGCCCAT | |
| | | | | | | | | | | | | | GTTATA | |
| | | | | | | | | | | | | | G-3′ | |
| H | oTLD21 | - | Human | hg38 | Chr | 2 | : | 111126335 | 111126358 | | 24 | 37 | 5′-dCGCA | AscI |
| | new | | | | | | | | | | | | TGGCGCGC | |
| | | | | | | | | | | | | | CCTACTTT | |
| | | | | | | | | | | | | | ACTTCACA | |
| | | | | | | | | | | | | | GGTATAAC | |
| | | | | | | | | | | | | | C-3′ | |
| I | oTLD33 | + | Mouse | mm10 | Chr | 2 | : | 128129549 | 128129573 | 843 | 25 | 38 | 5′-dCGCA | AscI |
| | | | | | | | | | | | | | TGGCGCGC | |
| | | | | | | | | | | | | | CGTAGAAT | |
| | | | | | | | | | | | | | TTTTCTAA | |
| | | | | | | | | | | | | | AACTATAT | |
| | | | | | | | | | | | | | TC-3′ | |
| J | oTLD32 | - | Mouse | mm10 | Chr | 2 | : | 128130340 | 128130367 | | 28 | 39 | 5′-dCGCA | SalI |
| | | | | | | | | | | | | | TGTCGACG | |
| | | | | | | | | | | | | | TATTAAGA | |
| | | | | | | | | | | | | | CTCTAATA | |
| | | | | | | | | | | | | | GCTTCCAG | |
| | | | | | | | | | | | | | AGG-3′ | |
| K | oTLD98 | + | Human | hg38 | Chr | 2 | : | 111127109 | 111127130 | 1441 | 22 | 35 | 5′-dCGCA | NotT |
| | | | | | | | | | | | | | TGCGGCCG | |
| | | | | | | | | | | | | | CTCTCCTT | |
| | | | | | | | | | | | | | ACACTCTG | |
| | | | | | | | | | | | | | GGAGGA | |
| | | | | | | | | | | | | | T-3′ | |
| L | oTLD10 | - | Human | hg38 | Chr | 2 | : | 111128507 | 111128525 | | 19 | 30 | 5′-dCGCA | BamHI |
| | | | | | | | | | | | | | TGGATCCA | |
| | | | | | | | | | | | | | ACAGCATG | |
| | | | | | | | | | | | | | ATGGTTCC | |
| | | | | | | | | | | | | | CC-3′ | |
| M | oTLD11 | + | Human | hg38 | Chr | 2 | : | 111128526 | 111128545 | 501 | 20 | 31 | 5′-dCGCA | EcoRI |
| | | | | | | | | | | | | | TGAATTCC | |
| | | | | | | | | | | | | | TCCATAGA | |
| | | | | | | | | | | | | | GGCTGTGC | |
| | | | | | | | | | | | | | CAT-3′ | |
| N | oTLD12 | - | Human | hg38 | Chr | 2 | : | 111128985 | 111129004 | | 20 | 31 | 5′-dCGCA | SalI |
| | | | | | | | | | | | | | TGTCGACT | |
| | | | | | | | | | | | | | GAGTGGGA | |
| | | | | | | | | | | | | | AGAGTCAA | |
| | | | | | | | | | | | | | GCC-3′ | |
| O | oTLD113 | + | Mouse | mm10 | Chr | 2 | : | 128147195 | 128147214 | 478 | 20 | 33 | 5′-dCGCA | NotI |
| | | | | | | | | | | | | | TGCGGCCG | |
| | | | | | | | | | | | | | CGTAAGGA | |
| | | | | | | | | | | | | | CCTCTCCC | |
| | | | | | | | | | | | | | CATCC-3′ | |
| P | oTLD114 | - | Mouse | mm10 | Chr | 2 | : | 128147618 | 128147646 | | 20 | 33 | 5′-dCGCA | AscI |
| | | | | | | | | | | | | | TGGCGCGC | |
| | | | | | | | | | | | | | CCCAACAG | |
| | | | | | | | | | | | | | GACAGCCA | |
| | | | | | | | | | | | | | GCTAC-3′ | |
| Q | oTLD115 | + | Human | hg38 | Chr | 2 | : | 111151266 | 111151285 | 842 | 20 | 33 | 5′-dCGCA | AscI |
| | | | | | | | | | | | | | TGGCGCGC | |
| | | | | | | | | | | | | | CGTGACTG | |
| | | | | | | | | | | | | | CTTCCGCT | |
| | | | | | | | | | | | | | AAAGG-3′ | |
| R | oTLD116 | - | Human | hg38 | Chr | 2 | : | 111152064 | 111152083 | | 20 | 31 | 5′-dCGCA | EcoRI |
| | | | | | | | | | | | | | TGAATTCC | |
| | | | | | | | | | | | | | TCCCCACT | |
| | | | | | | | | | | | | | TTGATCCT | |
| | | | | | | | | | | | | | GAA-3′ | |
| S | oTLD117 | + | Mouse | mm10 | Chr | 2 | : | 128147689 | 128147710 | 510 | 22 | 33 | 5′-dCGCA | BamHI |
| | | | | | | | | | | | | | TGGATCCG | |
| | | | | | | | | | | | | | CATCTTCA | |
| | | | | | | | | | | | | | GAAGCAGT | |
| | | | | | | | | | | | | | GTGTT-3′ | |
| T | oTLD118 | - | Mouse | mm10 | Chr | 2 | : | 128148155 | 128148176 | | 22 | 33 | 5′-dCGCA | SalI |
| | | | | | | | | | | | | | TGTCGACT | |
| | | | | | | | | | | | | | CCTCAGTC | |
| | | | | | | | | | | | | | CATTCATC | |
| | | | | | | | | | | | | | AACAG-3′ | |
| Y | oTLD40 | + | Mouse | mm10 | Chr | 2 | : | 128173955 | 128173974 | 388 | 20 | 31 | 5′-dCGCA | HinDIII |
| | | | | | | | | | | | | | TAAGCTTA | |
| | | | | | | | | | | | | | TCAGGCCC | |
| | | | | | | | | | | | | | AGGGTTCT | |
| | | | | | | | | | | | | | AGT-3′ | |
| Z | oTLD41 | - | Mouse | mm10 | Chr | 2 | : | 128174299 | 128174318 | | 20 | 33 | 5′-dCGCA | NotI |
| | | | | | | | | | | | | | TGCGGCCG | |
| | | | | | | | | | | | | | CATAGTGT | |
| | | | | | | | | | | | | | GCCTGTCC | |
| | | | | | | | | | | | | | CAAGG-3′ | |

Restriction enzyme sites have been incorporated within 11- to 13-base segments of non-homology at the 5′ end of each primer.

TABLE 2
Single Guide RNAs (sgRNAs)

| | Guide | Guide | Guide | PAM | Guide | Design | PAM | | mm 10 | |
|---|---|---|---|---|---|---|---|---|---|---|
| End | name | Rank | sequence | Sequence | length | parameter | location | Chr | coordinates | Strand |
| 5′ | BC1L1 | Guide #1 | AGTTGTACCA | TGG | 20 | TOP SCORE | UPSTREAM | Chr 2: | 128129677- | minus |
| | | | GGCATCACCG | | | | | | 128129696 | |
| 5′ | BC1R1 | Guide #5 | AAAATATCCA | TGG | 20 | TOP SCORE | DOWNSTREAM | Chr 2: | 128129667- | plus |
| | | | CGGTGATGCC | | | | | | 128129686 | |
| 5′ | BC1L3 | Guide #6 | TGTGGAAGTG | AGG | 20 | CLOSEST | UPSTREAM | Chr 2: | 128129686- | minus |
| | | | GACGAGTTTG | | | TO END | | | 128129625 | |
| 5′ | BC1R3 | Guide #10 | ACAACTTTTC | TGG | 20 | CLOSEST | DOWNSTREAM | Chr 2: | 128129623- | plus |
| | | | CCAGATCAGT | | | TO END | | | 128129642 | |
| 3′ | BC2L1 | Guide #1 | TACGTGGAGA | AGG | 20 | TOP SCORE | UPSTREAM | Chr 2: | 128147456- | minus |
| | | | AGCACCTTAC | | | | | | 128147475 | |
| 3′ | BC2R1 | Guide #4 | TGTAAGGTGC | AGG | 20 | TOP SCORE | DOWNSTREAM | Chr 2: | 128147455- | plus |
| | | | TTCTCCACGT | | | | | | 128147474 | |
| 3′ | BC2L3 | Guide #20 | TTATTTAAAT | AGG | 20 | CLOSEST | UPSTREAM | Chr 2: | 128147642- | minus |
| | | | AAATACCAAC | | | TO END | | | 128147661 | |
| 3′ | BC2R3 | Guide #14 | AGGGTAGCTG | TGG | 20 | CLOSEST | DOWNSTREAM | Chr 2: | 128147624- | plus |
| | | | GCTGTCCTGT | | | TO END | | | 128147643 | |

Four guides were designed at each end of the mouse Bcl2l11 gene segment to be replaced including two (one in each orientation) with the top design score, and two (one in each orientation) located closest to the outermost ends.

TABLE 3
Microinjection Mixes

| Experiment | Number | 1 | 2 | 3 | 4 | 5 | 6 | 7 | 8 |
|---|---|---|---|---|---|---|---|---|---|
| Guides | BC1L1 | | | 50 ng/μL | 50 ng/μL | | | 50 ng/μL | 50 ng/μL |
| | BC1R1 | | | 50 ng/μL | 50 ng/μL | | | 50 ng/μL | 50 ng/μL |
| | BC1L3 | 50 ng/μL | 50 ng/μL | | | 50 ng/μL | 50 ng/μL | | |
| | BC1R3 | 50 ng/μL | 50 ng/μL | | | 50 ng/μL | 50 ng/μL | | |
| | BC2L1 | | | 50 ng/μL | 50 ng/μL | | | 50 ng/μL | 50 ng/μL |
| | BC2R1 | | | 50 ng/μL | 50 ng/μL | | | 50 ng/μL | 50 ng/μL |
| | BC2L3 | 50 ng/μL | 50 ng/μL | | | 50 ng/μL | 50 ng/μL | | |
| | BC2R3 | 50 ng/μL | 50 ng/μL | | | 50 ng/μL | 50 ng/μL | | |
| Other | Cas9 mRNA | 100 ng/μL | 100 ng/μL | 100 ng/μL | 100 ng/μL | 100 ng/μL | 100 ng/μL | 100 ng/μL | 100 ng/μL |
| Reagents | Donor vector | 5 ng/μL | 1 ng/μL | 5 ng/μL | 1 ng/μL | 10 ng/μL | 5 ng/μL | 10 ng/μL | 5 ng/μL |
| Embryos | Rep 1 | ~80 | ~80 | ~80 | ~80 | ~80 | | ~80 | |
| Injected | Rep 2 | | | | | | ~80 | | ~80 |

Microinjection mixes contained four guides (either those with the highest scores or those with the most terminal positions within the mouse Bcl2l11 segment to be replaced) and varying concentrations of donor DNA (1, 5, or 10 ng/μL).

TABLE 4
Genotyping Oligonucleotides

| | Sequence | Length | Forward/ | | Coordinates |
|---|---|---|---|---|---|
| Primer | (5′ to 3′) | (nt) | Reverse | Chromosome | (mm10/hg38) |
| TLD56 | dATCTGTGGCCTTCTAGCCAA | 20 | Forward | Mouse Chr 2 | 128129240-128129259 |
| TLD57 | dAGAATGCCCTAACTCAGCCA | 20 | Reverse | Mouse Chr 2 | 128130445-128130464 |
| TLD239 | dTGCATCTAAGGGTTTGGCTT | 20 | Forward | Mouse Chr 2 | 128147294-128147313 |
| TLD338 | dGAGTCAAAGCCTACATCCCCAA | 22 | Reverse | Mouse Chr 2 | 128147780-128147801 |
| TLD335 | dGGAACAGCAAGTCGATCAACAC | 22 | Reverse | Human Chr 2 | 111125334-111125355 |
| TLD337 | dGGTGTTTGAGGAGAGTGCTGTA | 22 | Forward | Human Chr 2 | 111151728-111151749 |

Standard PCR primers were designed to amplify the junctions flanking the original mouse Bcl2l11 allele, the humanized BCL2L11 allele, and the deletion-bearing allele.

TABLE 5
Summary of the CRISPR-Stimulated Replacement of 17-Kilobase
Pairs of Mouse Bcl2l11 with 25-Kilobase Pairs of Human BCL2l11

| Experi- | Guide | | | | | | | |
|---|---|---|---|---|---|---|---|---|
| ment | Sets | [Donor] | P₆5¹ | Percentage | PCR Results | N₁5 | Percentage | PCR Results |
| 1 | CLOSEST | 5 ng/μL | 12 | 100.00% | No deletion of 17 kbp | N/A | | |
| | TO ENDS | | | | mouse segment; | | | |
| | | | | | No mouse human | | | |
| | | | | | junctions | | | |
| 2 | | 1 ng/μL | 1 | 12.50% | Positive for 5' and 3' | 50 | 100.0% | No deletion of 17-kbp |
| | | | | | mouse/human | | | mouse segment; |
| | | | | | junctions | | | No mouse |
| | | | | | | | | human junctions |
| | | | 7 | 87.50% | No deletion of 17-kbp | N/A | | |
| | | | | | mouse segment; | | | |
| | | | | | No mouse human | | | |
| | | | | | junctions | | | |
| 3 | HIGHEST | 5 ng/μL | 0 | N/A | | | | |
| | SCORE | | | | | | | |
| 4 | | 1 ng/μL | 16 | 100.00% | No deletion of 17-kbp | N/A | | |
| | | | | | mouse segment; | | | |
| | | | | | No mouse human | | | |
| | | | | | junctions | | | |
| 5 | CLOSEST | 10 ng/μL | 0 | N/A | | | | |
| | TO | 5 ng/μL | 1 | 5.56% | Deletion of 17-kbp | 14 | 45.2% | Positive for 5' and 3' |
| 6 | ENDS | | | | mouse segment; | | | mouse/human junctions |
| | | | | | Positive for 5' and 3' | 17 | 54.8% | Deletion of 17-kbp |
| | | | | | mouse/human | | | mouse segment |
| | | | | | junctions | | | |
| | | | 17 | 94.44% | No deletion of 17-kbp | N/A | | |
| | | | | | mouse segment; | | | |
| | | | | | No mouse human | | | |
| | | | | | junctions | | | |
| 7 | HIGHEST | 10 ng/μL | 1 | 14.29% | Deletion of 17-kbp | 14 | 25.0% | Deletion of 17-kbp |
| | SCORE | | | | mouse segment; | | | mouse segment |
| | | | | | Positive for 5' and 3' | 42 | 75.0% | No deletion of 17-kbp |
| | | | | | mouse/human | | | mouse segment; |
| | | | | | junctions | | | No mouse human |
| | | | | | | | | junctions |
| | | | 5 | 85.71% | No deletion of 17-kbp | N/A | | |
| | | | | | mouse segment; | | | |
| | | | | | No mouse human | | | |
| | | | | | junction | | | |
| 8 | | 5 ng/μL | 21 | 100.00% | No deletion of 17-kbp | N/A | | |
| | | | | | mouse segment; | | | |
| | | | | | No mouse human | | | |
| | | | | | junctions | | | |
| Experi- | | | | | F₁s | | | |
| ment | N₁s | N₂S | Percentage | PCR Results | crossed to | N₂S | Percentage | PCR Results |
| 1 | | | | | | | | |
| 2 | | | | | | | | |
| 3 | | | | | | | | |
| 4 | | | | | | | | |
| 5 | | | | | | | | |
| 6 | X FVB/NJ | 23 | 35.4% | Positive for 5' | X C57BL/6J | 3 | 33.3% | Positive for 5' |
| | (to generate | | | and 3' mouse/ | | | | and 3' mouse/ |
| | B6FVBFls) | | | human junction | | | | human junction |
| | | | | | | 6 | 66.7% | Wildtype |
| | | | | | X C57BL/GJ | 9 | 56.3% | Positive for 5' |
| | | | | | | | | and 3' mouse/ |
| | | | | | | | | human junctions |
| | | | | | | 7 | 43.8% | Wildtype |
| | | | | | X FVB/NJ | 7 | TBD | Not yet genotyped |
| | | | | | X FVB/NJ | 19 | TBD | Not yet genotyped |
| | | 42 | 64.6% | Wildtype | | | | |
| | X C57BL/6J | 10 | 43.5% | Positive for 5' | | | | |
| | | | | and 3' mouse/ | | | | |
| | | | | human Junctions | | | | |
| | | 13 | 56.5% | Wildtype | | | | |
| | X C57BL/6J | 9 | 36.0% | Positive for 5' | | | | |
| | | | | and 3' mouse/ | | | | |
| | | | | human junctions | | | | |
| | | 16 | 64.0% | Wildtype | | | | |
| | X C57BL/6J | 10 | 55.6% | Deletion of 17-kbp | | | | |
| | | | | mouse segment; | | | | |
| | | | | No mouse human | | | | |
| | | | | junctions | | | | |
| | | 8 | 44.4% | Wildtype | | | | |
| | X C57BL/6J | 6 | 37.5% | Deletion of 17-kbp | | | | |
| | | | | mouse segment; | | | | |
| | | | | No mouse human | | | | |
| | | | | junctions | | | | |
| | | 10 | 62.5% | Wildtype | | | | |
| | X C57BL/6J | 7 | 38.3% | Deletion of 17-kbp | | | | |
| | | | | mouse segment; | | | | |
| | | | | No mouse human | | | | |
| | | | | junctions | | | | |
| | | 11 | 61.1% | Wildtype | | | | |
| | X C57BL/6J | 9 | 56.3% | Deletion of 17-kbp | | | | |
| | | | | mouse segment; | | | | |
| | | | | No mouse human | | | | |
| | | | | junctions | | | | |
| | | 7 | 43.8% | Wildtype | | | | |
| 7 | | | | | | | | |
| 8 | | | | | | | | |

1. A method of inserting a large exogenous genomic DNA via homologous recombination to replace an endogenous genomic DNA in the genome of a cell of a mammal, comprising the steps of:
(a) providing a bacterial artificial chromosome (BAC);
(b) providing a large exogenous genomic DNA of about 10-300 kb;
(c) inserting said large exogenous genomic DNA into said BAC,
wherein said large exogenous genomic DNA is flanked by a proximal region of about 10-30 kb, and a distal region of about 10-30 kb, and
wherein said proximal region and said distal region flank said endogenous genomic DNA in the genome of said cell;
(d) preparing a first pair of CRISPR/Cas9 guide RNAs (gRNAs), said first pair comprises a first gRNA and a second gRNA, wherein said first gRNA and said second gRNA target a first Cas9 cleavage site and a second Cas9 cleavage site, respectively, in the endogenous genomic DNA, within about 250 bp from a proximal junction where said proximal region joins said endogenous genomic DNA in the genome of the cell;
(e) preparing a second pair of CRISPR/Cas9 guide RNAs (gRNAs), said second pair comprises a third gRNA and a fourth gRNA, wherein said third gRNA and said fourth gRNA target a third Cas9 cleavage site and a fourth Cas9 cleavage site, respectively, in the endogenous genomic DNA, within about 250 bp from the distal junction where said distal region joins said endogenous genomic DNA in the genome of the cell;
(f) providing a Cas9 protein, or a Cas9 coding sequence capable of producing the Cas9 protein; and
(g) introducing into said cell of said mammal:
(i) said BAC in step (c);
(ii) said first pair of CRISPR/Cas9 guide RNAs in step (d);
(iii) said second pair of CRISPR/Cas9 guide RNAs in step (e); and
(iv) said Cas9 protein or Cas9 coding sequence in step (f);
whereby:
(i) said first pair of gRNAs directs said Cas9 protein to cleave said first and said second Cas9 cleavage sites in said endogenous genomic DNA at the proximal junction to generate a first double-strand break (DSB);
(ii) said second pair of gRNAs directs said Cas9 protein to cleave said third and said fourth Cas9 cleavage sites in said endogenous genomic DNA at the distal junction to generate a second DSB; and
(iii) said large exogenous genomic DNA is integrated into the genome of the cell at said first DSB and said second DSB via homologous recombination to replace said endogenous genomic DNA between the proximal region and the distal region.

2. The method of claim 1, wherein said large exogenous genomic DNA is about 15-200 kb, about 20-100 kb, or about 25 kb.

3-4. (canceled)

5. The method of claim 1, wherein said cell is a zygote.

6. The method of claim 5, wherein step (g) is performed by microinjection.

7. The method of claim 6, where microinjection is performed using about 1-10 ng/μL, about 2-8 ng/μL, or about 5 ng/μL of said BAC containing said large exogenous genomic DNA.

8-9. (canceled)

10. The method of claim 1, wherein said cell is an embryonic stem (ES) cell.

11. The method of claim 10, wherein step (g) is performed by electroporation.

12. The method of claim 1, wherein said BAC carries no selection marker.

13. The method of claim 1, wherein said large exogenous genomic DNA is from a different strain of the same species of said mammal.

14. The method of claim 1, wherein said large exogenous genomic DNA is from a different species of said mammal.

15. The method of claim 1, wherein said mammal is a mouse.

16. The method of claim 1, wherein said first and said second Cas9 cleavage sites are independently within about 100 bp, 50 bp, or 10 bp from the proximal junction.

17. The method of claim 1, wherein said third and said fourth Cas9 cleavage sites are independently within about 100 bp, 50 bp, or 10 bp from the distal junction.

18. The method of claim 1, wherein said first gRNA and said second gRNA bind to different strands of the endogenous genomic DNA.

19. The method of claim 1, wherein said third gRNA and said fourth gRNA bind to different strands of the endogenous genomic DNA.

20. The method of claim 1, wherein said first and said second Cas9 cleavage sites are the two potential Cas9 cleavage sites closest to the proximal junction.

21. The method of claim 1, wherein said third and said fourth Cas9 cleavage sites are the two potential Cas9 cleavage sites closest to the distal junction.

22. The method of claim 1, wherein in step (f), said Cas9 protein is provided in a complex comprising said first gRNA, said second gRNA, said third gRNA, or said fourth gRNA.

23. A method of generating a non-human mammal whose cells harboring a large exogenous genomic that have replaced an endogenous genomic DNA via homologous recombination, and capable of transmitting the large exogenous genomic DNA through germline, comprising the steps of:
(a) providing a bacterial artificial chromosome (BAC);
(b) providing a large exogenous genomic DNA of about 10-300 kb;
(c) inserting said large exogenous genomic DNA into said BAC,
wherein said large exogenous genomic DNA is flanked by a proximal region of about 10-30 kb, and a distal region of about 10-30 kb, and
wherein said proximal region and said distal region flank said endogenous genomic DNA in the genome of said mammal;
(d) preparing a first pair of CRISPR/Cas9 guide RNAs (gRNAs), said first pair comprises a first gRNA and a second gRNA, wherein said first gRNA and said second gRNA target a first Cas9 cleavage site and a second Cas9 cleavage site, respectively, in the endogenous genomic DNA, within about 250 bp from a proximal junction where said proximal region joins said endogenous genomic DNA in the genome of the mammal;
(e) preparing a second pair of CRISPR/Cas9 guide RNAs (gRNAs), said second pair comprises a third gRNA and a fourth gRNA, wherein said third gRNA and said fourth gRNA target a third Cas9 cleavage site and a fourth Cas9 cleavage site, respectively, in the endogenous genomic DNA, within about 250 bp from the distal junction where said distal region joins said endogenous genomic DNA in the genome of the mammal;
(f) providing a Cas9 protein, or a Cas9 coding sequence capable of producing the Cas9 protein; and
(g) introducing into a zygote of said mammal:
(i) said BAC in step (c);
(ii) said first pair of CRISPR/Cas9 guide RNAs in step (d);
(iii) said second pair of CRISPR/Cas9 guide RNAs in step (e); and
(iv) said Cas9 protein or Cas9 coding sequence in step (f);
(h) preparing a pseudopregnant female of the same species of the mammal;
(j) implanting said zygote into said pseudopregnant female to give birth to an offspring of the mammal.

24-45. (canceled)


SEQUENCE LISTING (36 sequences)

SEQ ID NO: 1 - DNA, 31 bp, Artificial Sequence
  source: /note=“Description of Artificial Sequence Synthetic primer”
cgcatactagttccatccggtcatttctctc

SEQ ID NO: 2 - DNA, 31 bp, Artificial Sequence
  source: /note=“Description of Artificial Sequence Synthetic primer”
cgcataagcttttttgcttggtccagattcc

SEQ ID NO: 3 - DNA, 35 bp, Artificial Sequence
  source: /note=“Description of Artificial Sequence Synthetic primer”
cgcatgcggccgcatagtttaataaccaccaggca

SEQ ID NO: 4 - DNA, 32 bp, Artificial Sequence
  source: /note=“Description of Artificial Sequence Synthetic primer”
cgcataagcttaactgactgtagccccagaaa

SEQ ID NO: 5 - DNA, 31 bp, Artificial Sequence
  source: /note=“Description of Artificial Sequence Synthetic primer”
cgcataagctttattgctcagagggtttgga

SEQ ID NO: 6 - DNA, 31 bp, Artificial Sequence
  source: /note=“Description of Artificial Sequence Synthetic primer”
cgcatggatcctgatttacctcactgaagcc

SEQ ID NO: 7 - DNA, 35 bp, Artificial Sequence
  source: /note=“Description of Artificial Sequence Synthetic primer”
cgcatgaattcggcaggcctttgcccatgttatag

SEQ ID NO: 8 - DNA, 37 bp, Artificial Sequence
  source: /note=“Description of Artificial Sequence Synthetic primer”
cgcatggcgcgccctactttacttcacaggtataacc

SEQ ID NO: 9 - DNA, 38 bp, Artificial Sequence
  source: /note=“Description of Artificial Sequence Synthetic primer”
cgcatggcgcgccgtagaattttctaaaaactatattc

SEQ ID NO: 10 - DNA, 39 bp, Artificial Sequence
  source: /note=“Description of Artificial Sequence Synthetic primer”
cgcatgtcgacgtattaagactctaatagcttccagagg

SEQ ID NO: 11 - DNA, 35 bp, Artificial Sequence
  source: /note=“Description of Artificial Sequence Synthetic primer”
cgcatgcggccgctctccttacactctgggaggat

SEQ ID NO: 12 - DNA, 30 bp, Artificial Sequence
  source: /note=“Description of Artificial Sequence Synthetic primer”
cgcatggatccaacagcatgatggttcccc

SEQ ID NO: 13 - DNA, 31 bp, Artificial Sequence
  source: /note=“Description of Artificial Sequence Synthetic primer”
cgcatgaattcctccatagaggctgtgccat

SEQ ID NO: 14 - DNA, 31 bp, Artificial Sequence
  source: /note=“Description of Artificial Sequence Synthetic primer”
cgcatgtcgactgagtgggaagagtcaagcc

SEQ ID NO: 15 - DNA, 33 bp, Artificial Sequence
  source: /note=“Description of Artificial Sequence Synthetic primer”
cgcatgcggccgcgtaaggacctctccccatcc

SEQ ID NO: 16 - DNA, 33 bp, Artificial Sequence
  source: /note=“Description of Artificial Sequence Synthetic primer”
cgcatggcgcgccccaacaggacagccagctac

SEQ ID NO: 17 - DNA, 33 bp, Artificial Sequence
  source: /note=“Description of Artificial Sequence Synthetic primer”
cgcatggcgcgccgtgactgcttccgctaaagg

SEQ ID NO: 18 - DNA, 31 bp, Artificial Sequence
  source: /note=“Description of Artificial Sequence Synthetic primer”
cgcatgaattcctccccactttgatcctgaa

SEQ ID NO: 19 - DNA, 33 bp, Artificial Sequence
  source: /note=“Description of Artificial Sequence Synthetic primer”
cgcatggatccgcatcttcagaagcagtgtgtt

SEQ ID NO: 20 - DNA, 33 bp, Artificial Sequence
  source: /note=“Description of Artificial Sequence Synthetic primer”
cgcatgtcgactcctcagtccattcatcaacag

SEQ ID NO: 21 - DNA, 31 bp, Artificial Sequence
  source: /note=“Description of Artificial Sequence Synthetic primer”
cgcataagcttatcaggcccagggttctagt

SEQ ID NO: 22 - DNA, 33 bp, Artificial Sequence
  source: /note=“Description of Artificial Sequence Synthetic primer”
cgcatgcggccgcatagtgtgcctgtcccaagg

SEQ ID NO: 23 - DNA, 23 bp, Artificial Sequence
  source: /note=“Description of Artificial Sequence Synthetic oligonucleotide”
agttgtaccaggcatcaccgtgg

SEQ ID NO: 24 - DNA, 23 bp, Artificial Sequence
  source: /note=“Description of Artificial Sequence Synthetic oligonucleotide”
aaaatatccacggtgatgcctgg

SEQ ID NO: 25 - DNA, 23 bp, Artificial Sequence
  source: /note=“Description of Artificial Sequence Synthetic oligonucleotide”
tgtggaagtggacgagtttgagg

SEQ ID NO: 26 - DNA, 23 bp, Artificial Sequence
  source: /note=“Description of Artificial Sequence Synthetic oligonucleotide”
acaacttttcccagatcagttgg

SEQ ID NO: 27 - DNA, 23 bp, Artificial Sequence
  source: /note=“Description of Artificial Sequence Synthetic oligonucleotide”
tacgtggagaagcaccttacagg

SEQ ID NO: 28 - DNA, 23 bp, Artificial Sequence
  source: /note=“Description of Artificial Sequence Synthetic oligonucleotide”
tgtaaggtgcttctccacgtagg

SEQ ID NO: 29 - DNA, 23 bp, Artificial Sequence
  source: /note=“Description of Artificial Sequence Synthetic oligonucleotide”
ttatttaaataaataccaacagg

SEQ ID NO: 30 - DNA, 23 bp, Artificial Sequence
  source: /note=“Description of Artificial Sequence Synthetic oligonucleotide”
agggtagctggctgtcctgttgg

SEQ ID NO: 31 - DNA, 20 bp, Artificial Sequence
  source: /note=“Description of Artificial Sequence Synthetic primer”
atctgtggccttctagccaa

SEQ ID NO: 32 - DNA, 20 bp, Artificial Sequence
  source: /note=“Description of Artificial Sequence Synthetic primer”
agaatgccctaactcagcca

SEQ ID NO: 33 - DNA, 20 bp, Artificial Sequence
  source: /note=“Description of Artificial Sequence Synthetic primer”
tgcatctaagggtttggctt

SEQ ID NO: 34 - DNA, 22 bp, Artificial Sequence
  source: /note=“Description of Artificial Sequence Synthetic primer”
gagtcaaagcctacatccccaa

SEQ ID NO: 35 - DNA, 22 bp, Artificial Sequence
  source: /note=“Description of Artificial Sequence Synthetic primer”
ggaacagcaagtcgatcaacac

SEQ ID NO: 36 - DNA, 22 bp, Artificial Sequence
  source: /note=“Description of Artificial Sequence Synthetic primer”
ggtgtttgaggagagtgctgta